(19) United States
(12) Patent Application Publication
(10) Pub. No.: US 2025/0236645 A1
(43) Pub. Date: Jul. 24, 2025

(54) MRNA EXPRESSION AND DELIVERY SYSTEMS

(71) Applicant: Iowa State University Research Foundation, Inc., Ames, IA (US)

(72) Inventors: David Verhoeven, Ames, IA (US); Wyatt Allen Miller, Ames, IA (US); Balaji Narasimhan, Ames, IA (US); Michael Kimber, Ames, IA (US); Douglas Jones, Ames, IA (US)

(21) Appl. No.: 18/836,113

(22) PCT Filed: Feb. 8, 2023
(86) PCT No.: PCT/US2023/062194

§ 371 Date: Aug. 6, 2024

Related U.S. Application Data
(60) Provisional application No. 63/267,703, filed on Feb. 8, 2022.

(51) Int. Cl. C07K 14/005; A61K 9/50; A61K 39/00; A61K 39/145; C12N 5/09

(52) U.S. Cl. CPC C07K 14/005; A61K 9/5031; A61K 9/5068; A61K 39/145; C12N 5/0693; A61K 2039/53; C12N 2760/16122; C12N 2760/16134

(57) ABSTRACT

This present disclosure provides RNA expression systems based on sequences from plant viruses, insect viruses, and flaviviruses that eliminate the need for expensive modifications. Methods to express and package an RNA polynucleotide without the need for in vitro transcription and lipid nanoparticles are provided. Also provided are methods to package an RNA polynucleotide using synthetic polyanhydride nanoparticles that are stable at room temperature and suitable for delivery by nasal spray.

### CROSS-REFERENCE TO RELATED APPLICATION

This application claims priority to provisional application U.S. Ser. No. 63/267,703, filed Feb. 8, 2022, which is hereby incorporated herein by reference in its entirety.

### SEQUENCE LISTING XML

The instant application contains a sequence listing, which has been submitted in XML file format by electronic submission and is hereby incorporated by reference in its entirety. The XML file, created on Feb. 7, 2023, is named P13653WO00.xml and is 58,819 bytes in size.

### BACKGROUND

An mRNA vaccine can be highly efficacious for viruses as demonstrated by those targeting SARS-COV-2. However, the current technology suffers from a number of drawbacks: (1) the need to modify mRNA by using modified rNTPs like pseudouridine and a cap structure [e.g., m⁷G(5′)ppp(5′)GpN] at the 5′ end, and a run of 50 or more contiguous adenosine residues at the 3′ end [poly(A) tail], all of which require expensive steps to manufacture; (2) a lack of thermostability outside of −20 to −80° C.; (3) lipid nanoparticles whose chemical makeup are known to cause deleterious immune responses; (4) complicated purification systems where up to 25% of the product is small RNAs that do not make protein and little is known about; and (5) requirement for delivery by intramuscular injection, which requires a trained medical professional.

### SUMMARY

RNA polynucleotides comprising a 5′ untranslated region (5′ UTR), a heterologous sequence encoding at least one polypeptide, and a 3′ untranslated region (3′ UTR), wherein the 5′ UTR or the 3′ UTR comprise a panicum mosaic virus-like cap independent translation enhancer or an exoribonuclease-resistant RNA (xrRNA) element are provided.

In certain embodiments, the 5′ UTR or the 3′ UTR is from Thin paspalum asymptomatic virus (TPAV), Tomato Bushy Stunt Virus (TBSV), Sweet clover necrotic mosaic virus (SCNMV), Red clover necrotic mosaic virus (RCNMV), or Opium poppy mosaic virus (OPMV). In certain embodiments, the RNA polynucleotide does not comprise a 5′ cap structure or a poly-A tail. In certain embodiments, the RNA polynucleotide does not comprise a modified nucleoside. In certain embodiments, the encoded polypeptide is an antigenic polypeptide. In certain embodiments, the polypeptides are a light chain and a heavy chain of an antibody.

Pharmaceutical compositions comprising any of the RNA polynucleotides disclosed herein, and a pharmaceutically acceptable excipient are provided.

Exosomes for delivery of any of the RNA polynucleotides disclosed herein are provided.

Stable polyanhydride compositions comprising a polyanhydride polymer and any of the RNA polynucleotides disclosed herein are provided. Also provided are polyanhydride compositions comprising a polyanhydride polymer and any of the exosomes disclosed herein.

RNA polynucleotides comprising a 5′ UTR, a heterologous multiple cloning site for insertion of a sequence encoding a polypeptide, and a 3′ UTR, wherein the 5′ UTR or the 3′ UTR comprise a panicum mosaic virus-like cap independent translation enhancer or an xrRNA element are provided.

Methods of producing a polypeptide of interest in a subject comprising administering to the subject any of the RNA polynucleotides, the pharmaceutical compositions, the polyanhydride compositions, or the exosomes disclosed herein are provided.

Methods of delivering an RNA polynucleotide to a subject comprising administering to the subject the any of the RNA polynucleotides, the pharmaceutical compositions, the polyanhydride compositions, or the exosomes disclosed herein are provided.

Methods of inducing an immune response in a subject comprising administering to the subject a composition comprising an RNA polynucleotide in an amount effective to produce an antigen-specific immune response in the subject, wherein the RNA polynucleotide comprises a 5′ UTR, a sequence encoding at least one antigenic polypeptide, and a 3′ UTR, wherein the 5′ UTR or the 3′ UTR comprise a panicum mosaic virus-like cap independent translation enhancer or an xrRNA element are provided.

Methods of delivering an antibody to a subject comprising administering to the subject a composition comprising an RNA polynucleotide, wherein the RNA polynucleotide comprises a 5′ UTR, a sequence encoding a light chain of an antibody, a sequence encoding a heavy chain of an antibody, at least one internal ribosome entry site (IRES), and a 3′ UTR, wherein the 5′ UTR or the 3′ UTR comprise a panicum mosaic virus-like cap independent translation enhancer or an xrRNA element are provided.

Methods for producing exosomes for delivery of an RNA polynucleotide comprising transforming a cell with a polynucleotide construct that expresses an RNA polynucleotide, wherein the RNA polynucleotide comprises a 5′ UTR, a sequence encoding at least one polypeptide, and a 3′ UTR, wherein the 5′ UTR or the 3′ UTR comprise a panicum mosaic virus-like cap independent translation enhancer or an xrRNA element; culturing the cell in a growth media, wherein exosomes comprising the RNA polynucleotide are released into the extracellular growth media; removing the cells from the growth media; and harvesting the exosomes comprising the RNA polynucleotide from the growth media are provided.

While multiple embodiments are disclosed, still other embodiments of the disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF THE FIGURES

The following drawings form part of the specification and are included to further demonstrate certain embodiments or various aspects of the disclosure. In some instances, embodiments of the disclosure can be best understood by referring to the accompanying figures in combination with the detailed description presented herein. The description and accompanying figures may highlight a certain specific example, or a certain aspect of the disclosure. However, one skilled in the art will understand that portions of the example or aspect may be used in combination with other examples or aspects of the disclosure.

FIG. 1 shows cap-independent translation and stability elements in 5′ and 3′ UTRs of the mRNA vaccine transcript. Bold black lines: UTRs flanking ORF (bold box) encoding reporter (firefly luciferase (Fluc) or mKATE2) or vaccine antigen. RNA secondary structures of translation/stability elements are shown as wire diagrams with curved dashed lines indicating pseudoknot base pairing. Straight dashed lines indicate approximate location in which the element is inserted, with alternative structures mapped to the same sites. Cap analogs added at 5′ end (curved arrow) are in box at upper left. Names of elements are as in text, with lengths indicated. A (60)-X-A (60) indicates two 60 A tracts interrupted by 1-6 non-A bases.

FIG. 2 shows expression after mRNA transfection of cap less and tailless mRNA. in vitro transcription (IVT) was used to generate mRNA by commercial kit and transfected using mRNA transfection reagent. Cells were examined 48 hours post-transfection. TPAV and xrRNA cassettes far outperformed b-actin and continued to express the reporter for at least 3 days.

FIG. 3 shows that polyanhydride nanoparticles confer room temperature stability for at least 2 days. xrRNA mRNA was added to particles. 5 mg of particles were then degraded by incubating in water for 3 hours, RNA added to lipid transfection reagent, and then added onto HELA cells. Cells were examined 48 hours later.

FIG. 4 shows that mRNA persists in polyanhydride nanoparticles. Nanoparticles (nano) containing xrRNA flanking mKATE were dissolved for 4 hours or held at room temperature for 26 hours and then dissolved for 4 hours in water. mRNA not in the nanoparticles (naked) was subjected to similar conditions. RNA was reverse transcribed and then amplified by PCR for 40 cycles. Clearly, the nanoparticles impart some thermostability to the mRNA.

FIG. 5 shows enhanced mRNA delivery by copolymer. A549 cells were transfected using equine HA mRNA (0.5 μg) with lipid or copolymer or with H3N8 virus. We then stained for HA followed by anti-mouse Dylight 555 (red). The lipid was an optimized liposome from Mirus Bio for mRNA transfections. The copolymer not only transfected more cells but the red appeared deeper possibly meaning more protein translated. Some cell loss in the copolymer is attributed to gelling at 37° C. on top of the cells and removal of the gel before staining. Transwells do not incur this loss. A negative control for the lipid (not shown) was also similar to the copolymer negative. One representative well is shown for both delivery methods of 12 wells tested for each. Arrows indicate HA positive cells.

FIG. 6 shows thermostability at room temperature in polyanhydride. RSV F mRNA was made and lyophilized with spermidine. 80 μg was held at room temp in a closed microtube while 100 μg was encased in polyanhydride 20:80 and held in the same manner. After 4 months, the mRNA was added to liposomes according to manufactures direction and placed on HELA cells or 3 mm of polyanhydride was crumbed and added to HELA cells overnight. Cells were then intracellularly stained for RSV proteins followed by anti-goat Alex 555. qRT-PCR results also confirm that only the polyanhydride-bound mRNA was intact after storage for so long.

FIG. 7 shows mRNA in extracellular vesicles (EVs) in transfected cells. A549/T7 polymerase cells were transfected mKATE/TPAV (5 μg) using DEA Dextran. Cells were then shocked with 10% glycerol and media removed. After 3 days, supernatant was harvested by centrifugation and 0.2 μM filtering twice. 100 μl of EVs were added to HELA cells (no T7) for 1 hour before washing and replacing media. Cells began to florescence after 48 hours gaining more intensity after 72 hours.

FIG. 8 shows EVs examined by electron microscopy. EVs were purified by ultracentrifugation (not shown) or by commercial kit. They were then subjected to scanning electron microscopy.

FIG. 9 shows EVs harvested from cells after DEA Dextran transfection. A549 T7 cells were transfected with empty or with TPAV mKATE PCR products in DEA Dextran carrier with glycerol shock. Supernatant was removed and cells allowed to make EVs in serum free media for 3 days. These were harvested, spun down, and examined on the nanosight microscope. The empty transfection, on the left, has small EVs sizes that are not uniform. To the right, cells that had the TPAV PCR product and transcribed mRNAs through their T7 polymerase had larger EVs in the typical size for mRNA+EVs. The concentration of the ones on the right were 109/ml in a total of 30 ml. These were observed after one week at 4° C. as EVs and their contents are quite stable.

FIG. 10 shows the use of self-cleaving cassettes. The polycistronic system using self-cleaving peptides resulted in high expression of both coding sequences.

FIG. 11 shows a comparison of additional mRNA constructs expressed in T7 BHK cells. 1 mg of DNA transfected at 18 hours post transfection is shown. SCNMV and TPAV with 3′ xrRNA gave the most favorable expression of mCherry.

FIG. 12 shows the hemagglutination inhibition (HAI) titer of mice vaccinated with rHA from H3N8 once (rHA/1 dose) or twice (rHA/2 doses) with Alum or with mRNA on the TPAV cassette for H3N8 HA in the correct (mRNA/1 dose) or reversed orientation (Reversed mRNA) in EVs or β-actin (UTR) cap and tailed H3N8 HA in liposomes (HK mRNA/1 dose).

FIG. 13 shows naked mRNA transfection. A549 cells without T7 were transfected with sham (left), a swine cassette expressing mCherry (middle), or commercial mRNA expressing mCherry (modified U/C, Arco cap, and tailed) (right) using Ribojuice liposomes. Cells were examined at 18 hours post transfection. mCherry over brightfield images are shown.

FIG. 14 shows mRNA stability in polyanhydride nanoparticles. The mRNA constructs placed in polyanhydrides exhibited resistance to degradation from heat after 7 days at 37° C. Traditional IVT generated mRNA did not have this level of thermostability.

FIG. 15A-B shows circular RNA construction. FIG. 15A is an image of a gel showing two constructs that have been circularized and then digested with RNAse R that degrades linear RNA. FIG. 15B is an image of a gel showing a control that demonstrates RNAse R does degrade linear RNA.

FIG. 16A-B shows TPAV single expression systems. FIG. 16A shows a TPAV mRNA cassette (SEQ ID NO: 1) that includes T7 promoter, 5′ UTR from TPAV, cloning sites (BamHI and Xho) flanking mKATE2 reporter gene, and 3′ UTR from TPAV. FIG. 16B shows a TPAV mRNA cassette (SEQ ID NO: 2), which is the same as SEQ ID NO: 1 but with an RNA protein binding domain between the Xho cloning site behind a mCherry reporter gene (rather than mKATE) and in front of the 3′ UTR, and T7 polymerase terminator.

FIG. 17 shows a mRNA cassette with xrRNA using Red clover necrotic mosaic virus (RCNMV) (SEQ ID NO: 3). The cassette includes T7 promoter, cloning sites flanking a mCherry reporter gene, 3′ UTR xrRNA sequence from RCNMV, and T7 polymerase terminator.

FIG. 18 shows a xrRNA leader, 5′TPAV UTR, 3′TPAV UTR mRNA cassette (SEQ ID NO: 17). The cassette includes T7 polymerase promoter, xrRNA from Zika virus, 5′UTR TPAV, CDS of mCherry reporter gene, RNA protein binding domain, TPAV 3′ UTR, T7 polymerase terminator, and cloning sites (AAGCT, CTCGAG, and CATATG).

FIG. 19 shows a xrRNA, Cricket paralysis virus IRES, 3′ UTR TPAV mRNA expression cassette (SEQ ID NO: 18). The cassette includes T7 polymerase promoter, xrRNA from Zika virus, the IRES, ATGless CDS reporter, RNA protein binding domain, 3′ UTR TPAV, and T7 polymerase terminator.

FIG. 20 shows a xrRNA, TRIMV, 3′ TPAV UTR mRNA cassette (SEQ ID NO: 19). The cassette includes T7 polymerase promoter, xrRNA from Zika virus, TRIMV 5′IRES, CDS reporter, RNA protein binding domain, 3′ UTR TPAV, and T7 polymerase Termination terminator.

FIG. 21A-B shows mRNA cassettes based on Tomato bushy stunt virus (TBSV). FIG. 21A shows a cassette (SEQ ID NO: 20) that includes T7 polymerase promoter, T7 enhancer xrRNA from Zika virus, CDS reporter, RNA protein binding domain, TBSV 3′ UTR, T7 polymerase terminator, and cloning sites (AAGCT, CTCGAG, and CATATG). FIG. 21B shows a cassette (SEQ ID NO: 21) that includes T7 polymerase promoter, xrRNA from Zika virus, TBSV 5′ UTR, CDS reporter, TBSV 3′ UTR, and T7 polymerase terminator.

FIG. 22A-B shows a TPAV/head xrRNA cassette and a TPAV/Tail xrRNA cassette. FIG. 22A shows the TPAV/head xrRNA cassette (SEQ ID NO: 22) that includes T7 polymerase promoter, xrRNA from Red clover necrotic mosaic virus (RCNMV), mCherry reporter gene, 3′ UTR from TPAV. FIG. 22B shows the TPAV/Tail xrRNA cassette (SEQ ID NO: 23) that includes T7 polymerase promoter, 5′ UTR from RCNMV, mCherry reporter gene, xrRNA from RCNMV, and 3′ UTR from TPAV.

FIG. 23 shows a mRNA cassette based on RCNMV (SEQ ID NO: 24). The cassette includes T7 polymerase promoter, 5′ UTR from RCNMV, mCherry reporter gene, and 3′ UTR from RCNMV.

FIG. 24A-B shows dual expression systems. FIG. 24A shows a cassette for expression of antibodies (SEQ ID NO: 4). The cassettes contain heavy and light chains of antibodies without the CDR3 region flanked by the 3′UTR of xrRNA and a forward IRES from Polio virus and a back IRES from Cricket paralysis virus. FIG. 24B shows a cassette for vaccine manufacture (SEQ ID NO: 5), which is a similar construct but without the antibody genes. The forward CDS is based on eGFP and the back is based on mCherry. Both proteins can be cut out and replaced with a CDS of interest.

### BRIEF DESCRIPTION OF THE SEQUENCES

SEQ ID NOs: 1-5 and 17-24 are sequences of mRNA expression cassettes.

SEQ ID NO: 6 is a Zika virus xrRNA sequence.

SEQ ID NO: 7 is a TPAV 5′ UTR sequence.

SEQ ID NOs: 8 and 13 are TPAV 3′ UTR sequences.

SEQ ID NO: 9 is a Polio virus IRES sequence.

SEQ ID NO: 10 is a Cricket paralysis virus IRES sequence.

SEQ ID NO: 11 is an RNA protein binding domain sequence.

SEQ ID NO: 12 is a Triticum mosaic virus (TRIMV) IRES sequence.

SEQ ID NO: 14 is a Tomato bushy stunt virus (TBSV) 5′ UTR sequence.

SEQ ID NOs: 15 and 16 are TBSV 3′UTR sequences.

SEQ ID NOs: 25 and 36 are Red clover necrotic mosaic virus (RCNMV) xrRNA sequences.

SEQ ID NO: 26 and 37 are RCNMV 5′ UTR sequences.

SEQ ID NO: 27 and 38 are RCNMV 3′ UTR sequences.

SEQ ID NO: 28 is a Sweet clover necrotic mosaic virus (SCNMV) 5′ UTR sequence.

SEQ ID NO: 29 is a Classical swine fever virus 5′ UTR sequence.

SEQ ID NO: 30 is a Hepatitis C virus 3′ UTR sequence.

SEQ ID NO: 31 is a Potato leafroll virus xrRNA sequence.

SEQ ID NO: 32 is a Bovine viral diarrhea virus 5′ UTR sequence.

SEQ ID NO: 33 is an Opium poppy mosaic virus (OPMV) 5′ UTR sequence.

SEQ ID NO: 34 is a CHOP human mRNA sequence.

SEQ ID NO: 35 is a Zika virus 3′ UTR sequence.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 11/2, and 43/4 This applies regardless of the breadth of the range.

The term “about,” as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms.

As used herein, the term “biocompatible” means compatible with living cells, tissues, organs or systems posing little to no risk of injury, toxicity or rejection by the immune system.

As used herein, the term “biodegradable” means capable of being broken down into innocuous products by the action of living things.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

As used herein, the term “cyclic” refers to the presence of a continuous loop. Cyclic molecules need not be circular, only joined to form an unbroken chain of subunits. Cyclic molecules such as an RNA polynucleotide of the present disclosure may be single units or multimers or comprise one or more components of a complex or higher order structure.

As used herein, “delivery” refers to the act or manner of delivering a compound, substance, entity, moiety, cargo or payload.

As used herein, “delivery agent” refers to any substance which facilitates, at least in part, the in vivo delivery of an RNA polynucleotide to targeted cells.

As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

As used herein, the term “digest” means to break apart into smaller pieces or components. When referring to polypeptides or proteins, digestion results in the production of peptides.

As used herein, the term “distal” means situated away from the center or away from a point or region of interest.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, a “formulation” includes at least a polynucleotide and a delivery agent.

A “fragment,” as used herein, refers to a portion. For example, fragments of proteins may comprise polypeptides obtained by digesting full-length protein isolated from cultured cells.

As used herein, a “functional” biological molecule is a biological molecule in a form in which it exhibits a property and/or activity by which it is characterized.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a coding sequence is heterologous to an untranslated region, such as a 5′ UTR or 3′ UTR, on the same polynucleotide.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vitro synthesis” refers to an cell-free method of synthesis of mRNA.

As used herein, the term “in vivo” refers to events that occur within an organism (e.g., animal, plant, or microbe or cell or tissue thereof).

As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

A “multiple cloning site” (MCS) refers to nucleotide sequences comprising at least one unique restriction site, and, more typically, a grouping of unique restriction sites, for the purpose of cloning nucleic acid fragments into a vector or nucleic acid construct.

As used herein, “naturally occurring” means existing in nature without artificial aid.

As used herein, a “non human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

An “open reading frame” is a continuous stretch of nucleotides beginning with a start codon (e.g., methionine (ATG)), and ending with a stop codon (e.g., TAA, TAG or TGA) and encodes a polypeptide.

As used herein, the phrase “operably linked” refers to a functional connection between two or more molecules, constructs, transcripts, entities, moieties or the like.

As used herein, a “paratope” refers to the antigen-binding site of an antibody.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Examples of excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, the term “proximal” means situated nearer to the center or to a point or region of interest.

As used herein, “purify,” “purified,” “purification” means to make substantially pure or clear from unwanted components, material defilement, admixture or imperfection.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

As used herein “stable” refers to a compound that is sufficiently robust to survive isolation to a useful degree of purity from a reaction mixture, and preferably capable of formulation into an efficacious therapeutic agent.

As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans).

The term “therapeutic agent” refers to any agent that, when administered to a subject, has a therapeutic, diagnostic, and/or prophylactic effect and/or elicits a desired biological and/or pharmacological effect.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

### Nucleic Acids/Polynucleotides

Compositions, as provided herein, comprise at least one (one or more) ribonucleic acid (RNA) (e.g., mRNA) polynucleotide having an open reading frame encoding at least one polypeptide. The term “nucleic acid” includes any compound and/or substance that comprises a polymer of nucleotides (nucleotide monomer). These polymers are referred to as polynucleotides. Thus, the terms “nucleic acid” and “polynucleotide” are used interchangeably.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”'s in a representative DNA sequence but where the sequence represents RNA (e.g., mRNA), the “T”'s would be substituted for “U” s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features.

In certain embodiments, the RNA polynucleotide encodes at least two polypeptides. In some embodiments, an RNA polynucleotide encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 polypeptides. In some embodiments, an RNA polynucleotide encodes at least 10, 20, 30, 40, 50, 60, 70, 80, 90 or 100 polypeptides. In some embodiments, an RNA polynucleotide encodes at least 100 or at least 200 polypeptides. In some embodiments, an RNA polynucleotide encodes 1-10, 5-15, 10-20, 15-25, 20-30, 25-35, 30-40, 35-45, 40-50, 1-50, 1-100, 2-50 or 2-100 polypeptides.

In certain embodiments, the polynucleotides encoding the at least two polypeptides are separated from one another by a polynucleotide encoding a self-cleaving peptide. Self-cleaving peptides, first discovered in picornaviruses, are peptides of between 19 to 22 amino acids in length and are usually found between two proteins in some members of the picornavirus family. In some embodiments, the self-cleaving peptide is one or more 2A peptides. In some embodiments, the 2A self-cleaving peptide is selected from a F2A peptide (Foot-and-mouth disease virus 2A peptide), an E2A peptide (Equine rhinitis A virus 2A peptide), a P2A peptide (Porcine teschovirus-1 2A peptide), and a T2A peptide (Thosea asigna virus 2A).

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

A “5′ untranslated region” (5′UTR) refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

UTRs comprising a 3′ cap-independent translation enhancer derived from a plant virus and or an internal ribosome entry site are provided. Exonuclease-blocking structures at the 5′ end and virus-derived secondary structures at the 3′ end are also provided. The UTR elements circumvent the expense of capping, boost translation efficiency, and enhance RNA stability.

The UTR can comprise a cap-independent translation enhancer (CITE). Numerous small (˜100-150 nt) virus-derived CITEs in the 5′ region of plant viral 3′ UTRs that confer efficient translation on uncapped mRNA in plant cells have been characterized.

In certain embodiments, the CITE is a panicum mosaic virus-like CITE (PTE). The panicum mosaic virus-like 3′ CITE in the Thin paspalum asymptomatic virus (TPAV) genome facilitates very efficient translation of uncapped mRNA in mammalian cells. The PTE functions by binding translation initiation factor eIF4E which is well-conserved in 3D structure between plants and animals. This is novel because PTEs are the only known RNA structures lacking a m⁷G that bind tightly (Kd<100 nM) to eIF4E. eIF4E is part of the eIF4E/eIF4G/eIF4A heterotrimer known as eIF4F that is key for recruiting ribosomes to the 5′ cap of mRNA. In addition to binding eIF4F (via eIF4E), the PTE base pairs to the 5′ UTR of the mRNA, presumably to allow eIF4F to recruit the ribosome to the 5′ end. This circularizes the mRNA, forming a structure similar to that of the 5′ cap-eIF4F-PABP-poly(A) tail interaction on cellular mRNAs. Hence, in addition to obviating the need for a 5′ cap, PTE-mediated translation does not require a poly(A) tail. Instead, viruses that harbor a PTE (or any type of 3′ CITE) rely on viral structures downstream of the CITE to block 3′ to 5′ exonuclease activity.

In certain embodiments, the UTR comprises the natural TPAV 5′ and 3′ termini. This construct provides substantial expression in mammalian cells and the virus must have natural stability elements in order to prevent rapid degradation of its uncapped, non-polyadenylated RNA genome upon entering the cell. At the 3′ terminus, downstream of the 3′ CITE, all viruses in the TPAV family (Tombusviridae) harbor a bulged stem-loop, in which the bulged bases pair to the terminal GCCC, burying these bases in the middle of three coaxially stacked helices, likely hiding the 3′ end from exonuclease.

In certain embodiments, the UTR is derived from a virus of the family Tombusviridae. Examples of suitable viruses of the family Tombusviridae include, but are not limited to, Adonis mosaic virus, Angelonia flower break virus, Apple luteovirus 1, Apple-associated luteovirus, Artichoke mottled crinkle virus, Barley yellow dwarf virus, Bean leafroll virus, Beet black scorch virus, Calibrachoa mottle virus, Cardamine chlorotic fleck virus, Carnation Italian ringspot virus, Carnation mottle virus, Carnation ringspot virus, Carrot mottle mimic virus, Carrot mottle virus, Cherry associated luteovirus, Clematis chlorotic mottle virus, Cocksfoot mild mosaic virus, Cowpea mottle virus, Cucumber Bulgarian latent virus, Cucumber leaf spot virus, Cucumber necrosis virus, Cymbidium ringspot virus, Eggplant mottled crinkle virus, Elderberry aureusvirus 1, Elderberry latent virus, Ethiopian tobacco bushy top virus, Furcraea necrotic streak virus, Galinsoga mosaic virus, Grapevine Algerian latent virus, Groundnut rosette virus, Havel River virus, Hibiscus chlorotic ringspot virus, Honeysuckle ringspot virus, Ixeridium yellow mottle virus 2, Japanese iris necrotic ring virus, Jasmine virus H, Johnsongrass chlorotic stripe mosaic virus, Leek white stripe virus, Limonium flower distortion virus, Maize chlorotic mottle virus, Maize necrotic streak virus, Maize white line mosaic virus, Melon necrotic spot virus, Moroccan pepper virus, Neckar River virus, Nectarine stem pitting associated virus, Nootka lupine vein clearing virus, Oat chlorotic stunt virus, Olive latent virus 1, Olive mild mosaic virus, Opium poppy mosaic virus, Panicum mosaic virus, Patrinia mild mottle virus, Pea enation mosaic virus 2, Pea stem necrosis virus, Pelargonium chlorotic ring pattern virus, Pelargonium flower break virus, Pelargonium leaf curl virus, Pelargonium line pattern virus, Pelargonium necrotic spot virus, Pelargonium ringspot virus, Petunia asteroid mosaic virus, Potato necrosis virus, Pothos latent virus, Red clover necrotic mosaic virus, Rosa rugosa leaf distortion virus, Rose spring dwarf-associated virus, Saguaro cactus virus, Sitke waterborne virus, Soybean dwarf virus, Soybean yellow mottle mosaic virus, Sweet clover necrotic mosaic virus, Thin paspalum asymptomatic virus, Tobacco bushy top virus, Tobacco mottle virus, Tobacco necrosis virus A, Tobacco necrosis virus D, Tomato bushy stunt virus, Tralespevirus gompholobii, Tralespevirus lespedezae, Turnip crinkle virus, and Yam spherical virus. The UTR can comprise an IRES. In certain embodiments, the IRES is an intergenic region internal ribosome entry site (IGR IRES). These relatively compact (≤200 nt) structures allow extremely efficient internal initiation of translation of the second ORF in the genome of dicistroviruses, which infect invertebrates. They are much smaller than the >500 nt IRESes of type I, II, III IRESes of picorna and hepaciviruses, and likely stronger than host “IRESes” which occur only in 5′ UTRs on capped mRNAs and which usually function only under specific cell cycle stages or stress conditions. The X-ray crystal structure of the IGR IRES reveals that the 3′ portion of the structure mimics the anticodon stem-loop of a tRNA base paired to a codon in mRNA. This allows the IGR IRES to “fool” the 80S ribosome into “instant elongation” to translate the downstream RNA sequence, i.e. this translation requires no initiation factors and no start codon. This provides a key advantage in that host innate immune mechanisms such as PKR which block formation of the ribosomal pre-initiation complex should have no effect on IGR IRES-mediated translation.

Examples of IGR IRES sequences that can be used according to the present disclosure include without limitation, those from cricket paralysis virus (CrPV), aphid lethal paralysis virus, Rhopalosiphum padi virus (RhPV), and other dicistroviruses.

The UTR can comprise an element that further protects the RNA polynucleotide from exonucleolytic degradation.

In certain embodiments, the UTR comprises a tRNA-like structure (TLS) as present at the 3′end of many plant viral genomes. These multifunctional pseudoknotted structures interact with the host CTP: ATP nucleotidyl transferase that adds CCA to the 3′ ends of pre-tRNAs in a non-templated manner, thus serving as a telomerase to ensure intact 3′ ends. Host tRNA synthetases aminoacylate the 3′-end, just as they do on tRNAs, which further blocks exonucleases. The TLS is also known to enhance translation.

In certain embodiments, the UTR comprises a stable stem-loop structure as present at the 3′ ends of flaviviruses (e.g., Zika, West Nile viruses).

In certain embodiments, the UTR comprises an xrRNA sequence. These short (50-70 nt) but complex RNA structures have evolved to block and sequester the host exonuclease Xrn I, which is the primary nuclease for turnover of mRNAs after decapping. Xrn I also degrades certain RNA virus genomes in the 5′ to 3′ direction until it reaches the xrRNA structure at which it gets stuck, leaving a 3′ portion of the genome (usually the 3′ UTR) intact. xrRNAs are present in the 3′ UTRs of flaviviruses, generating noncoding, short flavivirus RNAs (sfRNAs) that serve a variety of proviral regulatory and immune suppressing functions.

Examples of suitable flaviviruses include, but are not limited to, Apoi virus, Aroa virus, Bagaza virus, Banzi virus, Bouboui virus, Bukalasa bat virus, Cacipacore virus, Carey Island virus, Cowbone Ridge virus, Dakar bat virus, Dengue virus, Edge Hill virus, Entebbe bat virus, Gadgets Gully virus, Ilheus virus, Israel turkey meningoencephalomyelitis virus, Japanese encephalitis virus, Japanese encephalitis virus group, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Modoc virus group, Montana myotis leukoencephalitis virus, mosquito-borne viruses, Murray Valley encephalitis virus, Ntaya virus, Ntaya virus group, Omsk hemorrhagic fever virus, Phnom Penh bat virus, Powassan virus, Rio Bravo virus, Rio Bravo virus group, Royal Farm virus, Saboya virus, Saint Louis encephalitis virus, Sal Vieja virus, San Perlita virus, Saumarez Reef virus, Seaborne tick-borne virus group, Sepik virus, Tembusu virus, Tick-borne encephalitis virus, Tyuleniy virus, Uganda S virus, Usutu virus, Wesselsbron virus, West Nile virus, Yaounde virus, Yellow fever virus, Yokose virus, and Zika virus.

In some embodiments, the xrRNA is positioned at the 5′ end of the 5′ UTR, immediately upstream of an IRES or the stem-loop complementary to a 3′ CITE. Because of its structure, it is possible that xrRNA could block ribosome entry and scanning at the 5′ end, as the 40S subunit prefers unstructured 5′ UTRs for optimal scanning to the start codon. This is not a problem for constructs with an IGR IRES because the ribosome enters directly at the IRES and does not scan, but CITE-mediated translation requires ribosome scanning from the 5′ end. xrRNA structures at sites predicted to be 5′ UTRs of subgenomic mRNAs that code for viral proteins are known, so it is expected that those xrRNAs would not block ribosome entry for translation. Given similarity of xrRNA structures of plant and animal viruses and the fact that yeast Xrn I is blocked by plant and animal viral xrRNAs, xrRNAs are predicted function in organisms of any kingdom.

In certain embodiments, the UTR comprises a polynucleotide that has at least 80%, at least 90%, at least 95%, at least 96%, at least 97%, at least 98%, at least 99%, or 100% sequence identity to SEQ ID NO: 6, 7, 8, 9, 10, 11, 12, 13, 14, 15, 16, 25, 26, 27, 28, 29, 30, 31, 32, 33, 34, 35, 36, 37, or 38.

The RNA polynucleotide of the present disclosure can comprise a 5′ cap. In certain embodiments, the RNA polynucleotide is uncapped (i.e. does not contain a 5′cap). The 5′-cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA. This 5′-guanylate cap may then be methylated to generate an N7-methyl-guanylate residue. The ribose sugars of the terminal and/or ante terminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

5′-cap structures include those described in International Patent Publication Nos. WO 2008016473 and WO 2011015347, each of which is incorporated herein by reference in its entirety.

The RNA polynucleotide of the present disclosure can comprise a poly-A tail. In certain embodiments, the RNA polynucleotide is non-polyadenylated (i.e. does not comprise a poly-A tail). During RNA processing, a long chain of adenosine nucleotides (poly-A tail) is normally added to a messenger RNA (mRNA) molecule to increase the stability of the molecule. Immediately after transcription, the 3′ end of the transcript is cleaved to free a 3′ hydroxyl. Then poly-A polymerase adds a chain of adenosine nucleotides to the RNA. The process, called polyadenylation, adds a poly-A tail that is between 100 and 250 residues long.

The RNA polynucleotide of the present disclosure can comprise a modified nucleoside. In certain embodiments, the RNA polynucleotide does not comprise a modified nucleoside. A “nucleoside” refers to a compound containing a sugar molecule (e.g., a pentose or ribose) or a derivative thereof in combination with an organic base (e.g., a purine or pyrimidine) or a derivative thereof (also referred to as a “nucleobase”). A “nucleotide” refers to a nucleoside with a phosphate group. Modified nucleotides may by synthesized by any useful method, such as, for example, chemically, enzymatically, or recombinantly, to include one or more modified or non-natural nucleosides.

### Polypeptides

Polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain polypeptides or multichain polypeptides, such as antibodies, and may be associated or linked to each other. Most commonly, disulfide linkages are found in multichain polypeptides. The term “polypeptide” may also apply to amino acid polymers in which at least one amino acid residue is an artificial chemical analogue of a corresponding naturally-occurring amino acid.

A “polypeptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

“Orthologs” refers to genes in different species that evolved from a common ancestral gene by speciation. Normally, orthologs retain the same function in the course of evolution. Identification of orthologs is important for reliable prediction of gene function in newly sequenced genomes.

“Analogs” is meant to include polypeptide variants that differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini and any combination(s) thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide-based or polynucleotide-based molecules.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that includes a stretch of 20, 30, 40, 50, or 100 (contiguous) amino acids that are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al. (1997).” Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12, 387 (1984)), BLASTP, BLASTN, and FASTA Altschul, S. F. et al., J. Molec. Biol., 215, 403 (1990)).

The polypeptide can be an antigenic polypeptide. In certain embodiment, the antigenic polypeptide is a viral or bacterial antigen. In some embodiments, the virus is a strain of adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome coronavirus; Severe acute respiratory syndrome coronavirus 2; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabiá virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Coronavirus; Japanese encephalitis virus; Vesicular exanthernavirus; and Eastern equine encephalitis.

In certain embodiments, the virus is a strain of Influenza A or Influenza B or combinations thereof. In some embodiments, the strain of Influenza A or Influenza B is associated with birds, pigs, horses, dogs, humans or non-human primates. In some embodiments, the antigenic polypeptide encodes a hemagglutinin protein or fragment thereof. In some embodiments, the hemagglutinin protein is H1, H2, H3, H4, H5, H6, H7, H8, H9, H10, H11, H12, H13, H14, H15, H16, H17, H18, or a fragment thereof.

In some embodiments, a composition comprises at least one polynucleotide having an open reading frame encoding a HA protein or immunogenic fragment thereof (e.g., at least one of H1-H18), a NP protein or immunogenic fragment thereof, a NA protein or immunogenic fragment thereof, a M1 protein or immunogenic fragment thereof, a M2 protein or immunogenic fragment thereof, a NS1 protein or immunogenic fragment thereof, and a NS2 protein or immunogenic fragment thereof, obtained from influenza virus.

The present disclosure encompasses compositions comprising multiple RNA polynucleotides, each encoding a single antigenic polypeptide, as well as compositions comprising a single RNA polynucleotide encoding more than one antigenic polypeptide (e.g., as a fusion polypeptide). Thus, a composition comprising an RNA polynucleotide having an open reading frame encoding a first antigenic polypeptide and an RNA polynucleotide having an open reading frame encoding a second antigenic polypeptide encompasses (a) compositions that comprise a first RNA polynucleotide encoding a first antigenic polypeptide and a second RNA polynucleotide encoding a second antigenic polypeptide, and (b) compositions that comprise a single RNA polynucleotide encoding a first and second antigenic polypeptide (e.g., as a fusion polypeptide). Compositions of the present disclosure, in some embodiments, comprise 2-10 (e.g., 2, 3, 4, 5, 6, 7, 8, 9 or 10), or more, RNA polynucleotides having an open reading frame, each of which encodes a different antigenic polypeptide (or a single RNA polynucleotide encoding 2-10, or more, different antigenic polypeptides).

A single RNA polynucleotide can encode two polypeptides. In certain embodiments, the two polypeptides comprise a light chain and a heavy chain of an antibody. As used herein, the term “antibody” encompasses both intact antibody and antibody fragment. Typically, an intact “antibody” is an immunoglobulin that binds specifically to a particular antigen. An antibody may be a member of any immunoglobulin class, including any of the human classes: IgG, IgM, IgE, IgA, and IgD. Typically, an intact antibody is a tetramer. Each tetramer is composed of two identical pairs of polypeptide chains, each pair having one “light” (approximately 25 kD) and one “heavy” chain (approximately 50-70 kD). The N-terminus of each chain defines a variable region of about 100 to 110 or more amino acids primarily responsible for antigen recognition. The terms “variable light chain” (VL) and “variable heavy chain” (VH) refer to these corresponding regions on the light and heavy chain respectively. Each variable region can be further subdivided into hypervariable (HV) and framework (FR) regions. The hypervariable regions comprise three areas of hypervariability sequence called complementarity determining regions (CDR 1, CDR 2 and CDR 3), separated by four framework regions (FR1, FR2, FR2, and FR4) which form a beta-sheet structure and serve as a scaffold to hold the HV regions in position. The C-terminus of each heavy and light chain defines a constant region consisting of one domain for the light chain (CL) and three for the heavy chain (CH1, CH2 and CH3). A light chain of immunoglobulins can be further differentiated into the isotypes kappa and lambda.

In some embodiments, the terms “intact antibody” or “fully assembled antibody” are used in reference to an antibody that contains two heavy chains and two light chains, optionally associated by disulfide bonds as occurs with naturally-produced antibodies. In some embodiments, an antibody according to the present disclosure is an antibody fragment.

As used herein, an “antibody fragment” includes a portion of an intact antibody, such as, for example, the antigen-binding or variable region of an antibody. Examples of antibody fragments include Fab, Fab′, F(ab′)₂, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains a sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)₂ fragment, scFv fragment, Fv fragment, dsFv diabody, dAb fragment, Fd′ fragment, Fd fragment, and an isolated complementarity determining region (CDR).

Any antibody known in the art can be used and antibodies can be produced against desired antigens using standard methods. The antibodies may be monoclonal antibodies, polyclonal antibodies, antibody mixtures or cocktails, human or humanized antibodies, chimeric antibodies, or bi-specific antibodies.

Exemplary antibodies include, but are not limited to, anti-chemokine (C-C motif) ligand 2 (CCL2), anti-lysyl oxidase-like-2 (LOXL2), anti-Flt-1, anti-TNF-α, anti-Interleukin-2Rα receptor (CD25), anti-TGFβ, anti-B-cell activating factor, anti-alpha-4 integrin, anti-BAGE, anti-β-catenin/m, anti-Bcr-abl, anti-C5, anti-CA125, anti-CAMEL, anti-CAP-1, anti-CASP-8, anti-CD4, anti-CD19, anti-CD20, anti-CD22, anti-CD25, anti-CDC27/m, anti-CD 30, anti-CD33, anti-CD52, anti-CD56, anti-CD80, anti-CDK4/m, anti-CEA, anti-CT, anti-CTL4, anti-Cyp-B, anti-DAM, anti-EGFR, anti-ErbB3, anti-ELF2M, anti-EMMPRIN, anti-EpCam, anti-ETV6-AML1, anti-HER2, anti-G250, anti-GAGE, anti-GnT-V, anti-Gp100, anti-HAGE, anti-HER-2/neu, anti-HLA-A*0201-R170I, anti-IGF-1R, anti-IL-2R, anti-IL-5, anti-MC1R, anti-myosin/m, anti-MUC1, anti-MUM-1,-2,-3, anti-proteinase-3, anti-p190 minor bcr-abl, anti-Pml/RARa, anti-PRAMS, anti-PSA, anti-PSM, anti-PSMA, anti-RAGE, anti-RANKL, anti-RU1 or RU2, anti-SAGE, anti-SART-1 or anti-SART-3, anti-survivin, anti-TEL/AML1, anti-TPI/m, anti-TRP-1, anti-TRP-2, anti-TRP-2/INT2, and anti-VEGF or anti-VEGF receptor.

### Therapeutic and Prophylactic Compositions

Provided herein are compositions (e.g., pharmaceutical compositions), methods, kits and reagents for prevention, treatment or diagnosis of infectious disease in humans and other animals. Compositions can be used as therapeutic or prophylactic agents. They may be used in medicine to prevent and/or treat infectious disease.

In some embodiments, compositions containing RNA polynucleotides as described herein can be administered to a subject (e.g., a mammalian subject, such as a human subject), and the RNA polynucleotides are translated in vivo to produce an antigenic polypeptide.

The compositions may be induced for translation of a polypeptide (e.g., antigen or immunogen) in a cell, tissue or organism. In some embodiments, such translation occurs in vivo, although such translation may occur ex vivo, in culture or in vitro. In some embodiments, the cell, tissue or organism is contacted with an effective amount of a composition containing an RNA polynucleotide that has at least one a translatable region encoding an antigenic polypeptide.

An “effective amount” of a composition is provided based, at least in part, on the target tissue, target cell type, means of administration, physical characteristics of the polynucleotide and other components, and other determinants. In general, an effective amount of the composition provides an induced or boosted immune response as a function of antigen production in the cell. Increased antigen production may be demonstrated by increased cell transfection (the percentage of cells transfected with the RNA), increased protein translation from the polynucleotide, decreased nucleic acid degradation (as demonstrated, for example, by increased duration of protein translation from a modified polynucleotide), or altered antigen specific immune response of the host cell.

In some embodiments, compositions (including polynucleotides their encoded polypeptides) in accordance with the present disclosure may be used for treatment of infectious disease.

Compositions may be administered prophylactically or therapeutically as part of an active immunization scheme to healthy individuals or early in infection during the incubation phase or during active infection after onset of symptoms. In some embodiments, the amount of composition of the present disclosure provided to a cell, a tissue or a subject may be an amount effective for immune prophylaxis.

Compositions may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In some embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, compositions may be administered intramuscularly, intradermally, or intranasally

Compositions may be utilized in various settings depending on the prevalence of the infection or the degree or level of unmet medical need. As a non-limiting example, the compositions may be utilized to treat and/or prevent a variety of infectious diseases.

Provided herein are pharmaceutical compositions optionally in combination with one or more pharmaceutically acceptable excipients.

Compositions may be formulated or administered alone or in conjunction with one or more other components. For instance, compositions (e.g., vaccine compositions) may comprise other components including, but not limited to, adjuvants. In some embodiments, compositions do not include an adjuvant (they are adjuvant free).

Compositions may be formulated or administered in combination with one or more pharmaceutically-acceptable excipients. In some embodiments, compositions comprise at least one additional active substances, such as, for example, a therapeutically-active substance, a prophylactically-active substance, or a combination of both. Compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, compositions are administered to humans, human patients or subjects. For the purposes of the present disclosure, the phrase “active ingredient” generally refers to the RNA polynucleotides (e.g., mRNA polynucleotides) encoding a polypeptide.

Formulations of the compositions described herein may be prepared by any method known or hereafter developed in the art of pharmacology. In general, such preparatory methods include the step of bringing the active ingredient (e.g., RNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

Compositions can be formulated using one or more excipients to: increase stability; increase cell transfection; permit the sustained or delayed release (e.g., from a depot formulation); alter the biodistribution (e.g., target to specific tissues or cell types); increase the translation of encoded protein in vivo; and/or alter the release profile of encoded protein (antigen) in vivo. Traditional excipients include any and all solvents, dispersion media, diluents, or other liquid vehicles, dispersion or suspension aids, surface active agents, isotonic agents, thickening or emulsifying agents, and preservatives.

In some embodiments, the compositions of the present disclosure can be used as a method of preventing an infection (e.g., a viral infection) in a subject, the method comprising administering to said subject at least one composition as provided herein. In some embodiments, the compositions of the present disclosure can be used as a method of treating an infection (e.g., a viral infection) in a subject, the method comprising administering to said subject at least one composition as provided herein. In some embodiments, the compositions of the present disclosure can be used as a method of reducing an incidence of infection (e.g., a viral infection) in a subject, the method comprising administering to said subject at least one composition as provided herein. In come embodiments, the compositions of the present disclosure can be used as a method of inhibiting spread from a first subject infected to a second subject not infected, the method comprising administering to at least one of said first subject sand said second subject at least one composition as provided herein.

A method of eliciting an immune response in a subject is provided. The method involves administering to the subject comprising at least one RNA polynucleotide having an open reading frame encoding at least one antigenic polypeptide or an immunogenic fragment thereof, thereby inducing in the subject an immune response. In some embodiments, the method may comprise exosome-mediated delivery of an RNA polynucleotide.

A prophylactically effective dose is a therapeutically effective dose that prevents infection with the virus at a clinically acceptable level. In some embodiments the therapeutically effective dose is a dose listed in a package insert for the vaccine.

### Exosomal Compositions

In certain embodiments, the RNA polynucleotide can be encapsulated by an exosome. The terms “exosomes”, “micro-vesicles” and “extracellular vesicles” are herein used interchangeably. They refer to extracellular vesicles, which are generally of between 30 and 200 nm, for example in the range of 50-100 nm in size. In some embodiments, the extracellular vesicles can be in the range of 20-300 nm in size, for example 30-250 nm in size, for example 50-200 nm in size.

The phrase “encapsulated by an exosome,” or grammatical variations thereof is used interchangeably herein with the phrase “exosomal composition” to refer to exosomes whose lipid bilayer surrounds an RNA polynucleotide.

In certain embodiments, the present disclosure provides an exosome for use in delivering an RNA polynucleotide to a cell. The present disclosure also provides a pharmaceutical composition comprising an exosome.

The exosome can comprise an RNA polynucleotide for delivery into a cell. In some embodiments, the delivery may be performed ex vivo. In some embodiments, the delivery may be performed in vivo. In certain embodiments, the pharmaceutical composition is in a form suitable for injection.

A method for producing exosomes comprising an RNA polynucleotide is provided, the method comprising transforming a cell with a polynucleotide construct that expresses an RNA polynucleotide; culturing the cell in a growth media, wherein exosomes comprising the RNA polynucleotide are released into the extracellular growth media; removing the cells from the growth media; and harvesting the exosomes comprising the RNA polynucleotide from the growth media. In certain embodiments, the harvesting is by ultracentrifugation. The cell can be an A549 cell. In certain embodiments, the cells constitutively express T7 RNA polymerase and the polynucleotide construct comprises a T7 promoter.

The exosomes comprising the RNA polynucleotide that can be harvested and used to directly transfect other cells or packaged into polyanhydride nanoparticles.

### Polyanhydride Polymers

In some embodiments, compositions comprising an RNA polynucleotide are formulated in a nanoparticle. Traditional mRNA vaccinations, especially those in the most advanced clinical trials (i.e., Moderna or CureVac), are based on liposomal transfections of cells after subQ/IM injection of the vaccine. The mRNA will then transfect local cells near the site of injection or transfect antigen-presenting cells recruited to the site of injection (liposome vaccines are known to express in the liver rather than locally). The liposomes must protect against RNAses before transfection of permissive cells and must then be endocytosed into cells to have any chance to be made into protein although the true mechanism of endosome escape it not known but presumed to be some export pore.

The present disclosure provides alternatives to liposome-mediated vehicles that enhance the transfection efficiency, better protect from thermo-degradation on the shelf and within the body or increase the subsequent immune profiles after vaccination.

In some embodiments, compositions comprising an RNA polynucleotide are formulated in a polyanhydride particle.

The terms “polyanhydride particle” and “polyanhydride nanosphere” both refer to microparticles and nanoparticles made of polyanhydride polymers as described herein. The polyanhydride polymers of the particles are typically copolymers, such as random mixes of anhydride oligomers (condense prepolymers). The polyanhydride particle can be abbreviated as “PA particle”, which can be a microparticle or a nanoparticle. The nanoparticles can also be referred to as polyanhydride nanosphere (PANS).

The group “alkyl” refers to a linear or branched hydrocarbon radical or diradical that is optionally unsaturated and optionally substituted with functional groups as described herein. The alkyl group can contain 1 to about 20 carbon atoms. Typical alkyl groups include, but are not limited to, methyl, ethyl, propyl, isopropyl, butyl, iso-butyl, sec-butyl, tert-butyl, pentyl, 3-pentyl, hexyl, heptyl, octyl, or decyl. In one embodiment, alkyl is preferably (C₁-C₆)alkyl. In another embodiment, alkyl is preferably (C₁-C₄)alkyl.

In an embodiment where the alkyl group is unsaturated, the alkyl is an alkenyl group or an alkynyl group. Alkenyl can be, for example, vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. The alkenyl can be unsubstituted or substituted. Alkynyl can be, for example, ethynyl, 1-propynyl, 2-propynyl, 1-butynyl, 2-butynyl, 3-butynyl, 1-hexynyl, 2-hexynyl, 3-hexynyl, 1-octynyl, and the like. The alkynyl can be unsubstituted or substituted.

The term “aryl” refers to an aromatic hydrocarbon derived from a parent aromatic ring system. The aryl can be linked to another group at a saturated or unsaturated carbon atom of the parent ring system. The aryl group can have 6 to about 14 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or substituted as described herein.

The term “halo” refers to fluoro, chloro, bromo, and iodo. Similarly, the term “halogen” refers to fluorine, chlorine, bromine, and iodine.

The term “substituted” is intended to indicate that one or more (e.g., 1, 2, 3, 4, or 5; in some embodiments 1, 2, or 3; and in other embodiments 1 or 2) hydrogen atoms on the group indicated in the expression using “substituted” is replaced with a selection from the substituents described hereinbelow, or with a suitable group known to those of skill in the art, provided that the indicated substituted atom's normal valency is not exceeded, and that the substitution results in a stable compound. Suitable substituent groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, acylamino, nitro, difluoromethyl, trifluoromethyl, trifluoromethoxy, carboxy, carboxyalkyl, keto, thioxo, alkylthio, alkylsulfinyl, alkylsulfonyl, and cyano. The suitable substituent groups can also include, e.g., —X, —R, —OR, —SR, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR, —S(═O)₂OH, —S(═O)R, —S(═O)₂R, —OS(═O)₂OR, —S(═O)₂NR, —OP(═O)(OR)₂, —P(═O)(OR)₂, —P(═O)(OH)₂, —C(═O)R, —C(═O)X, —C(S)R, —C(O)OR, —C(S)OR, —C(O)SR, —C(S)SR, —C(O)NRR, —C(S)NRR, —C(NR)NRR, where each X is independently a halogen (“halo”): F, Cl, Br, or I; and each R is independently H, alkyl, aryl, heterocycle, or a protecting group; or cations or anions thereof. As would be readily understood by one skilled in the art, when a substituent is keto (i.e., ═O) or thioxo (i.e., ═S), or the like, then two hydrogen atoms on the substituted atom are replaced.

As to any of the above groups that contain one or more substituents, it is understood, of course, that such groups do not contain any substitution or substitution patterns which are sterically impractical and/or synthetically non-feasible. In addition, the compounds of this disclosure include all stereochemical isomers arising from the substitution of these compounds.

The term “diacid” refers to any group that contains two carboxylic acid (—C(═O)OH) groups. The diacid can be an aliphatic dicarboxylic acid or an aromatic dicarboxylic acid. An aliphatic dicarboxylic acid is any alkyl group that is substituted with two (or more) carboxylic acid groups. An aromatic dicarboxylic acid is any compound that contains an at least one aryl group and two (or more) carboxylic acids. The two carboxylic acid groups can be on the same aryl group or they can be on different aryl groups. When the two carboxylic acid groups are on different aryl groups, the aryl groups can be linked by a single bond, or then can be linked by other groups, for example, an alkyl group. The alkyl group linking the aryl groups can be optionally substituted and optionally interrupted between carbons with other groups as defined herein.

The term “polymer” refers to a molecule of one or more repeating monomeric residue units covalently bonded together by one or more repeating chemical functional groups. The term includes all polymeric forms such as linear, branched, star, random, block, graft and the like. It includes homopolymers formed from a single monomer, copolymers formed from two or more monomers, terpolymers formed from three or more polymers and other polymers formed from more than three monomers. Differing forms of a polymer may also have more than one repeating, covalently bonded functional group.

The term “polyanhydride” refers to a polymer that is derived from the condensation of carboxylic acids or carboxylic acid derivatives such that repeating units of the resulting polymer are linked by anhydride (—C(═O)—O—C(═O)—) groups. Polyanhydrides can be prepared by condensing diacids or by condensing anhydride prepolymers, as described herein.

The term “carboxylic anhydride” refers to a compound that contains an anhydride (—C(═O)—O—C(═O)—) group. A carboxylic anhydride typically contains only one anhydride group per molecule. Carboxylic anhydrides can be formed by the condensation of two carboxylic acids. Carboxylic anhydrides that can be used in conjunction with the methods described herein include bis-alkyl carboxylic anhydrides, bis-aryl carboxylic anhydrides, and mixed anhydrides. Examples include, but are not limited to acetic anhydride, trifluoroacetic anhydride, and benzoic anhydride. Mixed anhydrides can also be employed, such as acetic benzoic anhydride, which is the condensation product of acetic acid and benzoic acid.

As used herein, an “acyl” group is a group, such as a (C₁-C₄)alkyl group, that terminates in a carbonyl radical at its point of attachment to another group. An “acyloxy” group is a substituent, such as a (C₁-C₄)alkyl group, that terminates in a carboxyl radical at its point of attachment to another group.

The term “acylated” refers to the conversion of a hydroxyl group into an acyloxy group. Acylation can be carried out by contacting a hydroxyl group or hydroxyl-containing group with a carboxylic anhydride.

As used herein, a “prepolymer” is a monomer, oligomer, or mixture thereof that can be converted into a polymer (e.g., a longer chain polyanhydride). Diacid prepolymers are typically acylated on their terminal carboxy groups. A prepolymer can be, for example, a bis(carboxylic acid acetyl ester), or an anhydride oligomer thereof. In some embodiments, a prepolymer can be a 1,ω-(4-acetoxycarbonylphenoxy)alkane, or an anhydride oligomer thereof. The phenoxy group of the 1,ω-(4-acetoxycarbonylphenoxy)alkane can have ortho, meta, or para substitution patterns.

As used herein, a “homopolymer” is a polymer that is made up of repeating units of one type of monomer. A “copolymer” is a polymer that is made up of repeating units of two or more different types of monomers. In a random copolymer, the organization of the repeating units is random.

The polyanhydrides used to prepare the particles of the disclosure can be prepared as described herein or by methods known to those of skill in the art. A number of examples of methods for the preparation of polyanhydrides are provided below. A wide range of suitable diacids can be employed to prepare polyanhydrides. The diacid can be a diacid-substituted straight or branched chain alkane that is optionally interrupted by about one to about five -Ph-, —O—, —CH═CH—, and/or —N(R)—× groups wherein R is H, phenyl, benzyl, or (C₁-C₆)alkyl. In one embodiment, the alkane of the diacid can be C2-C12 (alkyl). In another embodiment, the alkane can be C4-C8 (alkyl). Additionally, the alkane group of the diacid can be optionally interrupted by about 1 to about 12 —OCH₂CH₂O— groups, for example, a poly(ethylene glycol) segment. The alkane group can also be optionally substituted with one, two, or three (C₁-C₆)alkyl, (C₁-C₆)alkenyl, trifluoromethyl, trifluoromethoxy, or oxo groups; or combinations thereof.

In one embodiment, a prepolymer can be prepared as illustrated in Scheme 1:

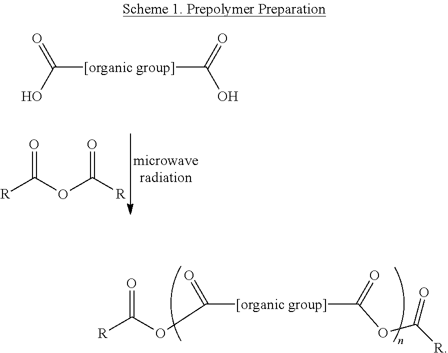

where “organic group” is any organic group that can link two carboxylic acid moieties, R is alkyl or aryl, and n is 1 to about 12. Examples of suitable organic groups include, but are not limited to, C₂-C₁₂(alkyl) groups, -PhO-C₂-C₁₂(alkyl)-OPh-groups, and PEG groups having 1 to about 12 PEG units, such as a 3,6-dioxaoctane group. A molar excess of the carboxylic anhydride can be employed. About 2 to about 30 molar equivalents of the carboxylic anhydride can be used. Alternatively, about 5 to about 20 molar equivalents of the carboxylic anhydride can be used. In one embodiment, 6 molar equivalents of the carboxylic anhydride are employed. In another embodiment, 18 molar equivalents of the carboxylic anhydride are employed. The carboxylic anhydride can be, for example, acetic anhydride, trifluoroacetic anhydride, benzoic anhydride, combinations thereof, and/or derivatives thereof.

A prepolymer can also be prepared as illustrated in Scheme 2:

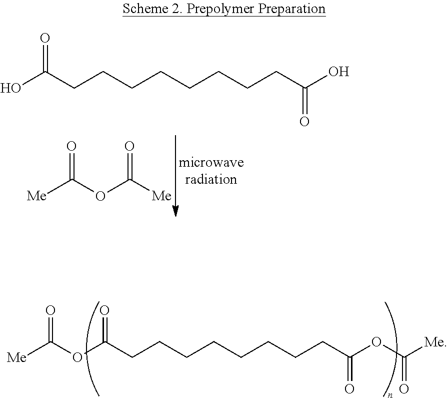

wherein n is 1 to about 12. Other carboxylic anhydrides can be used to form the end groups of the prepolymer, such as, but not limited to, benzoic anhydride. The central aliphatic group can optionally be substituted or interrupted as described herein.

The diacid can also be a 1, ω-bis(carboxy)alkane. As would be recognized by one skilled in the art, alternative nomenclature for a 1, ω-bis(carboxy)alkane is a 1,ω-alkanedioic acid that has two additional carbons in the alkane moiety compared to the corresponding bis(carboxy)alkane.

A prepolymer can also be prepared as illustrated in Scheme 3:

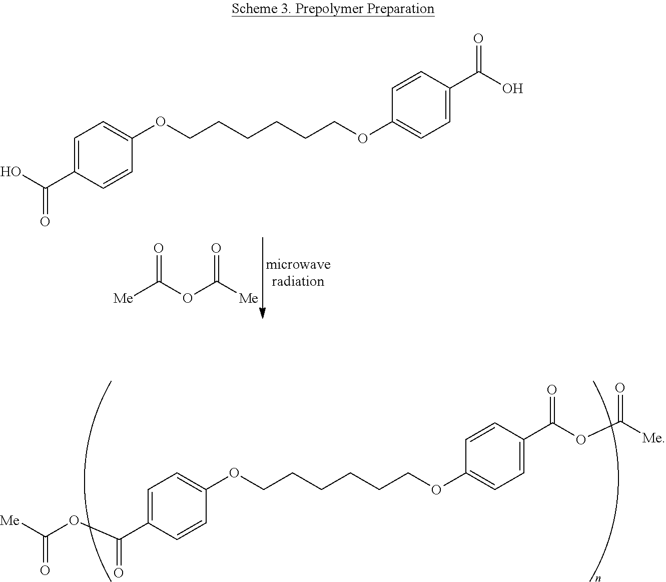

wherein n is 1 to about 12. Carboxylic anhydrides other than acetic anhydride can be used to form the end groups of the prepolymer. The central aliphatic group, the aryl groups, or both, can optionally be substituted, in any combination. The central aliphatic group can also be interrupted by oxygen, for examples, as with a poly(ethylene glycol) chain.

Accordingly, the diacid can be two aryl groups that are each substituted with a carboxy group wherein the aryl groups are linked by a straight or branched chain alkane that is optionally interrupted by about one to about five-Ph-, —O—, —CH═CH—, and/or —N(R)— groups wherein R is H, phenyl, benzyl, or (C₁-C₆)alkyl. In some embodiments, one or both of the aryl groups can be omitted and the carboxy groups are linked by the alkyl chain. In one embodiment, the alkane can be C₂-C₁₂ (alkyl). In another embodiment, the alkane can be C₄-C₈ (alkyl). In another embodiment, the alkane can be one or more PEG groups. Additionally, the alkane group linking the carboxylic acid-substituted aryl groups can be optionally interrupted by 1 to about 12 —OCH₂CH₂O— groups, for example, a poly(ethylene glycol) segment. The alkane group linking the carboxylic acid-substituted aryl groups can also be optionally substituted with one, two, or three (C₁-C₆)alkyl, (C₁-C₆)alkenyl, trifluoromethyl, trifluoromethoxy, or oxo groups; or combinations thereof.

The diacid can be a 1,ω-bis(4-carboxyphenoxy)alkane. In one embodiment, the alkane is a (C₂-C₁₀)alkane. In another embodiment, the alkane can be a C₄-C₈ (alkyl). In certain specific embodiments, alkane can be ethyl, propyl, butyl, pentyl, hexyl, heptyl, octyl, and branched isomers thereof. In one embodiment, the diacid is a 1,6-bis(4-carboxyphenoxy) hexane. In another embodiment, the diacid is a 1,6-bis(carboxy) octane. In another embodiment, the diacid can be a 1,8-bis(carboxyphenoxy)-3,6-dioxaoctane. Mixtures of any of these diacids can be used in conjunction with the microwave facilitated methods described herein.

Polyanhydrides can be prepared by condensation methods known in the art or by irradiating a prepolymer with a sufficient amount of microwave irradiation to polymerize the prepolymer. A sufficient amount of microwave radiation can typically be generated by a conventional microwave oven set to 1100 Watts for about 1 to about 30 minutes. More often, a sufficient amount of microwave radiation can be generated in about 1 to about 20 minutes. The resulting polyanhydride can be a homopolymer or a copolymer, depending on the nature of the prepolymer composition used in the reaction.

A polyanhydride can also be prepared by forming a prepolymer in situ from diacids. The diacids can be converted into prepolymers by irradiating diacids in the presence of a carboxylic anhydride. The prepolymer can be prepared by, for example, by irradiating a mixture of (a) a carboxylic anhydride and (b) an aromatic dicarboxylic acid, an aliphatic dicarboxylic acid, or a mixture thereof, with an amount of microwave radiation effective to form the prepolymer. One suitable carboxylic anhydride is acetic anhydride. Other suitable carboxylic anhydrides include, for example, trifluoroacetic anhydride and benzoic anhydride.

The terminal groups of polyanhydrides prepared according to the methods described herein will typically have terminal acyl groups. It is possible for some hydrolysis of the polyanhydrides to occur during the reaction or during the isolation of the polyanhydride. Thus, some terminal groups of such polyanhydrides can be carboxylic acid groups. Accordingly, the methods of the disclosure include the preparation of polyanhydrides that terminate in acyl groups, carboxylic acid groups, or combinations thereof.

The polyanhydride can be prepared, for example, as illustrated in Scheme 4:

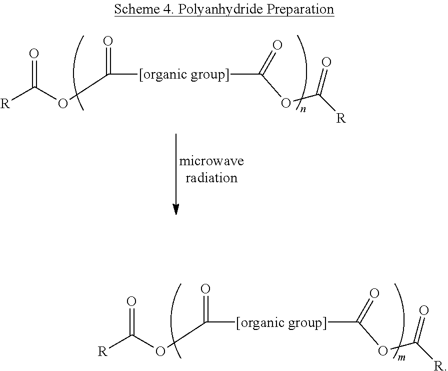

where “organic group” is any organic group that links two carboxylic acid moieties, R is alkyl or aryl, n is 1 to about 12, and m is about 5 to about 200.

The polyanhydride can also be prepared as illustrated in Scheme 5:

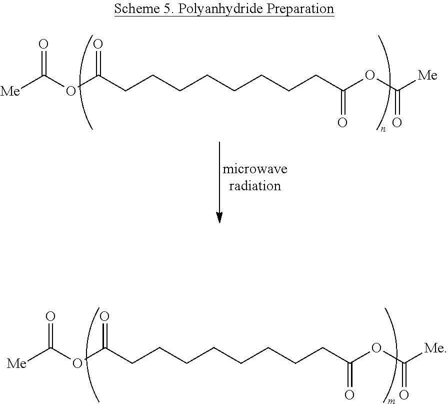

where n is 1 to about 12 and m is about 5 to about 200. In other embodiments, m can be about 10 to about 100, or about 10 to about 50. As would be understood by one skilled in the art, the value of m will typically be larger than the value of n. End groups other than acetate can be used and the central aliphatic group can be optionally substituted or optionally interrupted (e.g., as for PEG groups), or both, as described herein.

The polyanhydride can also be prepared as illustrated in Scheme 6:

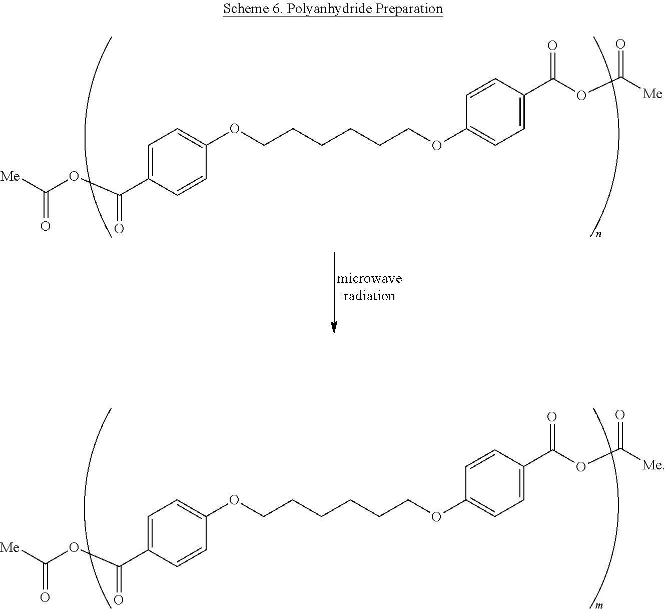

wherein n is 1 to about 12 and m is about 5 to about 100. In other embodiments, m can be about 10 to about 50, or about 15 to about 35. End groups other than acetate can be used and the central aliphatic group, the aryl groups, or both, can optionally be substituted, in any combination. The central aliphatic group can also be optionally interrupted by oxygen, for examples, as with a poly(ethylene glycol) chain.

A method for preparing the polyanhydride microparticles or nanoparticles includes irradiating one or more diacids, wherein the one or more diacids include an aromatic dicarboxylic acid, an aliphatic dicarboxylic acid, or a mixture thereof, with microwave radiation in the presence of a carboxylic anhydride so as to acylate one or more diacids to yield at least one prepolymer; and irradiating the prepolymer with microwave radiation so as to polymerize said prepolymer to yield the polyanhydride, as a homopolymer or a copolymer.

The prepolymers can be made up of dicarboxylic acids (“diacids”) that are acylated at both acid moieties. A prepolymer can be a single acylated diacid unit (monomer), or it can have up to about 12 condensed diacid units. A mixture of different diacids can be employed. The mixture of diacids can yield a random copolymer. The one or more diacids can include a diacid-substituted C₂-C₁₂ straight or branched chain alkane that is optionally interrupted by about 1 to about 5-Ph-, —O—, —CH═CH—, and/or —N(R)— groups wherein R is H, phenyl, benzyl, or (C₁-C₆)alkyl. The one or more diacids can also be optionally interrupted by about 1 to about 12 —OCH₂CH₂O— groups. The one or more diacids can also be optionally substituted with 1, 2, or 3 trifluoromethyl, trifluoromethoxy, (C₁-C₆)alkyl, (C₁-C₆)alkenyl, or oxo groups, or combinations thereof.

The at least one diacid can be a 1,0-bis(carboxy)alkane. The at least one diacid can also be a 1, ω-bis(4-carboxyphenoxy)alkane. The alkane can be, for example, a (C₃-C₈)alkane. Specific examples of the alkane include hexane and octane. The diacid can be 1,6-bis(4-carboxyphenoxy) hexane. Alternatively, the diacid can be 1,6-bis(carboxy) octane (sebacic acid). The at least one prepolymer can also include a bis(carboxylic acid acetyl ester), or an anhydride oligomer thereof. The at least one prepolymer can also include a 1, ω-(4-acetoxycarbonylphenoxyl)alkane, or an anhydride oligomer thereof, or a 1,8-bis(carboxyphenoxy)-3,6-dioxaoctane, or an anhydride oligomer thereof.

The carboxylic anhydride can be a bis-alkyl carboxylic anhydride, a bis-aryl carboxylic anhydride, an alkyl-aryl carboxylic anhydride, or a mixture thereof. The carboxylic anhydride can be, for example, acetic anhydride, trifluoroacetic anhydride, or benzoic anhydride. A molar excess of the carboxylic anhydride can be employed. Excess carboxylic anhydride can be removed after the prepolymer has formed.

In various embodiments, the polymers of the microparticles and/or nanoparticles described herein can be poly-sebacic anhydrides (SA), poly-1,6-bis-(p-carboxyphenoxy) hexane (CPH) anhydrides, or poly-1,8-bis(carboxyphenoxy)-3,6-dioxaoctane (CPTEG) anhydrides. In other embodiments, the polymers of the microparticles and/or nanoparticles described herein can be copolymers of sebacic anhydride (SA) and 1,6-bis-(p-carboxyphenoxy) hexane (CPH) anhydride, or copolymers of 1,8-bis(carboxyphenoxy)-3,6-dioxaoctane (CPTEG) anhydride and 1,6-bis-(p-carboxyphenoxy)hexane (CPH) anhydride. The ratio of SA to CPH, or CPTEG to CPH, can be any integer from about 1:19 to about 19:1. In certain embodiments, the ratio of CPTEG to CPH is about 20:80. An example of a structure of a SA:CPH copolymer is:

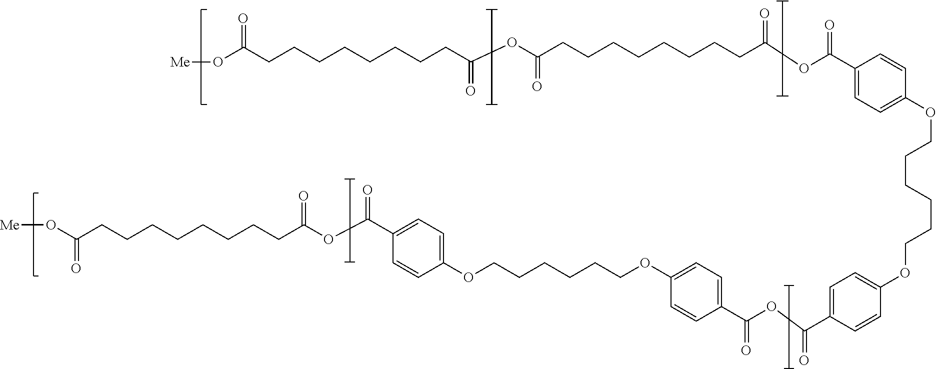

where each block (designated by a single or double bracket) includes a number of repeating units sufficient to provide a polymer with an Mₙ of about 5,000 to about 50,000 g/mol, such as about 10,000 to about 25,000 g/mol, or about 15,000 to about 20,000 g/mol. The anhydride copolymer can be a block copolymer or a random copolymer, or a combination thereof. CPTEG:CPH copolymers can also be prepared to form polymers where each block can include a number of repeating units sufficient to provide a polymer with an Mₙ of about 5,000 to about 50,000 g/mol, such as about 10,000 to about 25,000 g/mol, or about 15,000 to about 20,000 g/mol.

The polyanhydride particles described herein can be loaded with an effective amount of an RNA polynucleotide. In some embodiments, exosomes comprising an RNA polynucleotide can be encapsulated into polyanhydride polymers.

### Modes of Administration

Compositions may be administered by any route which results in a therapeutically effective outcome. These include, but are not limited, to intradermal, intramuscular, intranasal and/or subcutaneous administration. The present disclosure provides methods comprising administering compositions comprising an RNA polynucleotide to a subject in need thereof. The exact amount required will vary from subject to subject, depending on the species, age, and general condition of the subject, the severity of the disease, the particular composition, its mode of administration, its mode of activity, and the like. Compositions comprising an RNA polynucleotide are typically formulated in dosage unit form for ease of administration and uniformity of dosage. It will be understood, however, that the total daily usage of compositions comprising an RNA polynucleotide may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions comprising an RNA polynucleotide may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see, e.g., the range of unit doses described in International Publication No WO2013078199, the contents of which are herein incorporated by reference in their entirety). The desired dosage may be delivered three times a day, two times a day, once a day, every other day, every third day, every week, every two weeks, every three weeks, every four weeks, every 2 months, every three months, every 6 months, etc. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In certain embodiments, compositions comprising an RNA polynucleotide may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, compositions comprising an RNA polynucleotide may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, compositions comprising an RNA polynucleotide may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a composition comprising an RNA polynucleotide may be administered three or four times.

In some embodiments, compositions comprising an RNA polynucleotide may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

A composition comprising an RNA polynucleotide described herein can be formulated into a dosage form described herein, such as an intranasal, intratracheal, or injectable (e.g., intravenous, intraocular, intravitreal, intramuscular, intradermal, intracardiac, intraperitoneal, intranasal and subcutaneous).

### Embodiments

The following numbered embodiments also form part of the present disclosure:

1. An RNA polynucleotide comprising a 5′ untranslated region (5′ UTR), a heterologous sequence encoding at least one polypeptide, and a 3′ untranslated region (3′ UTR), wherein the 5′ UTR or the 3′ UTR comprise a cap independent translation enhancer or an exoribonuclease-resistant RNA (xrRNA) element.

2. The RNA polynucleotide of embodiment 1, wherein the 5′ UTR or the 3′ UTR comprises an xrRNA element.

3. The RNA polynucleotide of embodiment 1 or embodiment 2, wherein the xrRNA element comprises SEQ ID NO: 6, 25, 31, or 36.

4. The RNA polynucleotide of any one of embodiments 1-3, wherein the 5′ UTR or the 3′ UTR comprises a panicum mosaic virus-like cap independent translation enhancer.

5. The RNA polynucleotide of any one of embodiments 1-4, wherein the 5′ UTR or the 3′ UTR is from Thin paspalum asymptomatic virus (TPAV), Tomato Bushy Stunt Virus (TBSV), Sweet clover necrotic mosaic virus (SCNMV), Red clover necrotic mosaic virus (RCNMV), or Opium poppy mosaic virus (OPMV).

6. The RNA polynucleotide of any one of embodiments 1-5, wherein the 5′ UTR or the 3′ UTR comprises a polynucleotide that has at least 80%, at least 90%, at least 95%, at least 96%, at least 97%, at least 98%, at least 99%, or 100% sequence identity to SEQ ID NO: 6, 7, 8, 9, 10, 11, 12, 13, 14, 15, 16, 25, 26, 27, 28, 29, 30, 31, 32, 33, 34, 35, 36, 37, or 38.

7. The RNA polynucleotide of any one of embodiments 1-6, wherein the 5′ UTR comprises SEQ ID NO: 7, 14, 26, 28, 29, 32, 33, or 37, and wherein the 3′ UTR comprises SEQ ID NO: 8, 13, 15, 16, 27, 30, 35, or 38.

9. The RNA polynucleotide of any one of embodiments 1-7, wherein the RNA polynucleotide comprises an internal ribosome entry site (IRES).

9. The RNA polynucleotide of any one of embodiments 1-8, wherein IRES comprises SEQ ID NO: 9, 10, 12, or 34.

10. The RNA polynucleotide of any one of embodiments 1-9, wherein the RNA polynucleotide comprises an RNA protein binding domain.

11. The RNA polynucleotide of any one of embodiments 1-10, wherein the RNA polynucleotide does not comprise a 5′ cap structure or a poly-A tail.

12. The RNA polynucleotide of any one of embodiments 1-11, wherein the RNA polynucleotide does not comprise a modified nucleoside.

13. The RNA polynucleotide of any one of embodiments 1-12, wherein the at least one polypeptide comprises an antigenic polypeptide.

14. The RNA polynucleotide of embodiment 13, wherein the antigenic polypeptide is from an influenza virus or a coronavirus.

15. The RNA polynucleotide of any one of embodiments 1-12, wherein the at least one polypeptide comprises a light chain and a heavy chain of an antibody.

16. The RNA polynucleotide of any one of embodiments 1-15, wherein the RNA polynucleotide is a circular RNA polynucleotide.

17. A polyanhydride composition comprising a polyanhydride polymer and the RNA polynucleotide of any one of embodiments 1-16, wherein the polyanhydride polymer encapsulates the RNA polynucleotide.

18. The composition of embodiment 17, wherein the polyanhydride polymer comprises copolymers of 1,8-bis(carboxyphenoxy)-3,6-dioxaoctane (CPTEG) and 1,6-bis-(p-carboxyphenoxy) hexane (CPH).

19. The composition of embodiment 18, wherein the CPTEG:CPH ratio is about 20:80 to about 50:50.

20. An exosome for delivery of a RNA polynucleotide, the exosome comprising: an RNA polynucleotide comprising a 5′ UTR, a sequence encoding at least one polypeptide, and a 3′ UTR, wherein the 5′ UTR or the 3′ UTR comprise a cap independent translation enhancer or an xrRNA element.

21. The exosome of embodiment 20, wherein the 5′ UTR or the 3′ UTR comprises an xrRNA element.

22. The exosome of embodiment 20 or embodiment 21, wherein the xrRNA comprises SEQ ID NO: 6, 25, 31, or 36.

23. The exosome of any one of embodiments 20-22, wherein the 5′ UTR or the 3′ UTR comprises a panicum mosaic virus-like cap independent translation enhancer.

24. The exosome of any one of embodiments 20-23, wherein the 5′ UTR or the 3′ UTR is from TPAV, TBSV, SCNMV, RCNMV, or OPMV.

25. The exosome of any one of embodiments 20-24, wherein the 5′ UTR or the 3′ UTR comprises a polynucleotide that has at least 80%, at least 90%, at least 95%, at least 96%, at least 97%, at least 98%, at least 99%, or 100% sequence identity to SEQ ID NO: 6, 7, 8, 9, 10, 11, 12, 13, 14, 15, 16, 25, 26, 27, 28, 29, 30, 31, 32, 33, 34, 35, 36, 37, or 38.

26. The exosome of any one of embodiments 20-25, wherein the 5′ UTR comprises SEQ ID NO: 7, 14, 26, 28, 29, 32, 33, or 37, and wherein the 3′ UTR comprises SEQ ID NO: 8, 13, 15, 16, 27, 30, 35, or 38.

28. The exosome of any one of embodiments 20-26, wherein the RNA polynucleotide comprises an IRES.

29. The exosome of any one of embodiments 20-27, wherein IRES comprises SEQ ID NO: 9, 10, 12, or 34.

30. The exosome of any one of embodiments 20-28, wherein the RNA polynucleotide comprises an RNA protein binding domain.

31. The exosome of any one of embodiments 20-29, wherein the RNA polynucleotide does not comprise a 5′ cap structure or a poly-A tail.

32. The exosome of any one of embodiments 20-30, wherein the RNA polynucleotide does not comprise a modified nucleoside.

33. The exosome of any one of embodiments 20-31, wherein the at least one polypeptide comprises an antigenic polypeptide.

34. The exosome of embodiment 33, wherein the antigenic polypeptide is from an influenza virus or a coronavirus.

35. The exosome of any one of embodiments 20-34, wherein the at least one polypeptide comprises a light chain and a heavy chain of an antibody.

36. A polyanhydride composition comprising a polyanhydride polymer and the exosome of any one of embodiments 20-35, wherein the polyanhydride polymer encapsulates the exosome.

37. The composition of embodiment 36, wherein the polyanhydride polymer comprises copolymers of CPTEG and CPH.

38. The composition of embodiment 37, wherein the CPTEG:CPH ratio is about 20:80 to about 50:50.

39. A pharmaceutical composition comprising: the RNA polynucleotide of any one of embodiments 1-16; and a pharmaceutically acceptable excipient.

40. An RNA polynucleotide comprising a 5′ UTR, a heterologous multiple cloning site for insertion of a sequence encoding a polypeptide, and a 3′ UTR, wherein the 5′ UTR or the 3′ UTR comprise a cap independent translation enhancer or an xrRNA element.

41. A DNA polynucleotide encoding the RNA polynucleotide of any one of embodiments 1-16 or 40.

42. A method of producing a polypeptide of interest in a subject, the method comprising: administering to the subject the RNA polynucleotide of any one of embodiments 1-16; the composition of any one of embodiments 17-19 or 36-39; or the exosome of any one of embodiments 20-35.

43. A method of delivering an RNA polynucleotide to a subject, the method comprising: administering to the subject the RNA polynucleotide of any one of embodiments 1-16; the composition of any one of embodiments 17-19 or 36-39; or the exosome of any one of embodiments 20-35.

44. A method of inducing an immune response in a subject, the method comprising: administering to the subject a composition comprising an RNA polynucleotide in an amount effective to produce an antigen-specific immune response in the subject, wherein the RNA polynucleotide comprises a 5′ UTR, a sequence encoding at least one antigenic polypeptide, and a 3′ UTR, wherein the 5′ UTR or the 3′ UTR comprise a cap independent translation enhancer or an xrRNA element.

45. The method of embodiment 44, wherein the 5′ UTR or the 3′ UTR comprises an xrRNA element.

46. The method of embodiment 44 or embodiment 45, wherein the xrRNA element comprises SEQ ID NO: 6, 25, 31, or 36.

47. The method of any one of embodiments 44-46, wherein the 5′ UTR or the 3′ UTR comprises a panicum mosaic virus-like cap independent translation enhancer.

48. The method of any one of embodiments 44-47, wherein the 5′ UTR and/or the 3′ UTR is from TPAV, TBSV, SCNMV, RCNMV, or OPMV.

49. The method of any one of embodiments 44-48, wherein the 5′ UTR or the 3′ UTR comprises a polynucleotide that has at least 80%, at least 90%, at least 95%, at least 96%, at least 97%, at least 98%, at least 99%, or 100% sequence identity to SEQ ID NO: 6, 7, 8, 9, 10, 11, 12, 13, 14, 15, 16, 25, 26, 27, 28, 29, 30, 31, 32, 33, 34, 35, 36, 37, or 38.

50. The method of any one of embodiments 44-49, wherein the 5′ UTR comprises SEQ ID NO: 7, 14, 26, 28, 29, 32, 33, or 37, and wherein the 3′ UTR comprises SEQ ID NO: 8, 13, 15, 16, 27, 30, 35, or 38.

51. The method of any one of embodiments 44-50, wherein the RNA polynucleotide comprises an IRES.

52. The method of any one of embodiments 44-51, wherein IRES comprises SEQ ID NO: 9, 10, 12, or 34.

53. The method of any one of embodiments 44-52, wherein the RNA polynucleotide comprises an RNA protein binding domain.

54. The method of any one of embodiments 44-53, wherein the RNA polynucleotide does not comprise a 5′ cap structure or a poly-A tail.

55. The method of any one of embodiments 44-54, wherein the RNA polynucleotide does not comprise a modified nucleoside.

56. The method of any one of embodiments 44-55, wherein the antigenic polypeptide is from an influenza virus or a coronavirus.

57. The method of any one of embodiments 44-56, wherein the composition comprises a pharmaceutically acceptable excipient.

58. The method of any one of embodiments 44-57, wherein the composition comprises an exosome.

59. The method of any one of embodiments 44-58, wherein the composition comprises a polyanhydride polymer.

60. The method of embodiment 59, wherein the polyanhydride polymer comprises copolymers of CPTEG and CPH.

61. The method of embodiment 60, wherein the CPTEG:CPH ratio is about 20:80 to about 50:50.

62. The method of any one of embodiments 44-61, wherein the composition is administered to the subject intramuscularly, intranasally, or intradermally.

63. The method of any one of embodiments 44-62, wherein the composition is administered to the subject intranasally.

64. The method of any one of embodiments 44-63, wherein the subject is a human.

65. A method of delivering an antibody to a subject, the method comprising: administering to the subject a composition comprising an RNA polynucleotide, wherein the RNA polynucleotide comprises a 5′ UTR, a sequence encoding a light chain of an antibody, a sequence encoding a heavy chain of an antibody, at least one IRES, and a 3′ UTR, wherein the 5′ UTR or the 3′ UTR comprise a cap independent translation enhancer or an xrRNA element.

66. The method of embodiment 65, wherein the 5′ UTR or the 3′ UTR comprises an xrRNA element.

67. The method of embodiment 65 or embodiment 66, wherein the xrRNA element comprises SEQ ID NO: 6, 25, 31, or 36.

68. The method of any one of embodiments 65-67, wherein the 5′ UTR or the 3′ UTR comprises a panicum mosaic virus-like cap independent translation enhancer.

69. The method of any one of embodiments 65-68, wherein the 5′ UTR and/or the 3′ UTR is from TPAV, TBSV, SCNMV, RCNMV, or OPMV.

70. The method of any one of embodiments 65-69, wherein the 5′ UTR or the 3′ UTR comprises a polynucleotide that has at least 80%, at least 90%, at least 95%, at least 96%, at least 97%, at least 98%, at least 99%, or 100% sequence identity to SEQ ID NO: 6, 7, 8, 9, 10, 11, 12, 13, 14, 15, 16, 25, 26, 27, 28, 29, 30, 31, 32, 33, 34, 35, 36, 37, or 38.

71. The method of any one of embodiments 65-70, wherein the 5′ UTR comprises SEQ ID NO: 7, 14, 26, 28, 29, 32, 33, or 37 and wherein the 3′ UTR comprises SEQ ID NO: 8, 13, 15, 16, 27, 30, 35, or 38.

72. The method of any one of embodiments 65-71, wherein the RNA

polynucleotide comprises two IRESes, and wherein the IRESes comprise SEQ ID NO: 9 and SEQ ID NO: 10.

73. The method of any one of embodiments 65-72, wherein the RNA polynucleotide comprises an RNA protein binding domain.

74. The method of any one of embodiments 65-73, wherein the RNA polynucleotide does not comprise a 5′ cap structure or a poly-A tail.

75. The method of any one of embodiments 65-74, wherein the RNA polynucleotide does not comprise a modified nucleoside.

76. The method of any one of embodiments 65-75, wherein the composition comprises a pharmaceutically acceptable excipient.

77. The method of any one of embodiments 65-76, wherein the composition comprises an exosome.

78. The method of any one of embodiments 65-77, wherein the composition comprises a polyanhydride polymer.

79. The method of embodiment 78, wherein the polyanhydride polymer comprises copolymers of CPTEG and CPH.

80. The method of embodiment 79, wherein the CPTEG:CPH ratio is about 20:80 to about 50:50.

81. The method of any one of embodiments 65-80, wherein the composition is administered to the subject intramuscularly, intranasally, or intradermally.

82. The method of any one of embodiments 65-81, wherein the subject is a human.

83. A method for producing exosomes for delivery of an RNA polynucleotide, the method comprising: transforming a cell with a polynucleotide construct that expresses an RNA polynucleotide, wherein the RNA polynucleotide comprises a 5′ UTR, a sequence encoding at least one polypeptide, and a 3′ UTR, wherein the 5′ UTR or the 3′ UTR comprise a cap independent translation enhancer or an xrRNA element; culturing the cell in a growth media, wherein exosomes comprising the RNA polynucleotide are released into the extracellular growth media; removing the cells from the growth media; and harvesting the exosomes comprising the RNA polynucleotide from the growth media.

84. The method of embodiment 83, wherein the harvesting is by ultracentrifugation.

85. The method of embodiment 83 or embodiment 84, wherein the cell is an A549 cell.

86. The method of any one of embodiments 83-85, wherein the polynucleotide construct comprises a promoter.

87. The method of embodiment 86, wherein the promoter is a T7 promoter, and wherein the cells constitutively express T7 RNA polymerase.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The following examples are offered by way of illustration and not by way of limitation.

### EXAMPLES

### Example 1: Alternative mRNA Expression Cassettes

A series of expression cassettes that function to stabilize mRNA by flanking a coding region with untranslated regions that confer some protection from host endonucleases were tested. Expression of a fluorescent reporter flanked by beta-actin (housekeeping protein abundant in cells) untranslated regions (UTRs), similar to that on the Moderna vaccine mRNA, but without capping or tailing, was transient (<24 hrs), unless it was continuously being transcribed by an intracellular T7 RNA polymerase. In contrast, the fluorescent reporter protein persisted within the cells when using exonuclease-resistant RNA structures (xrRNA), and UTRs of Thin paspalum asymptomatic virus (TPAV), and aphid lethal paralysis virus (ALPV) flanking the coding region of the reporter gene, suggesting that the mRNA flanked by these UTRs persisted, and was translated efficiently, despite lacking a cap and a poly(A) tail.

The goal of mRNA vaccines is to express a protein (the antigen) within the cells so as to initiate a long-lived antibody response against the protein. These alternative expression cassettes appear to do that better than the traditional mRNA constructs containing “housekeeping gene” UTRs+cap+tailing. By far, the most favorable expression cassette tested to date, is that containing the UTRs from TPAV.

For specific methodology, in vitro transcription (IVT)-expressed mRNA was generated using a T7 RNA polymerase expression kit and mRNA was harvested by phenol/chloroform and ethanol/salt purification. 1 μg of mRNA was transfected into cells using Mirus Biotech's mRNA transfection reagent. A similar transfection using only a PCR fragment containing the T7 promoter, 5′ and 3′ UTRs, and the reporter coding region was also transfected by Lipofectamine into A549 cells that constituently express the T7 polymerase.

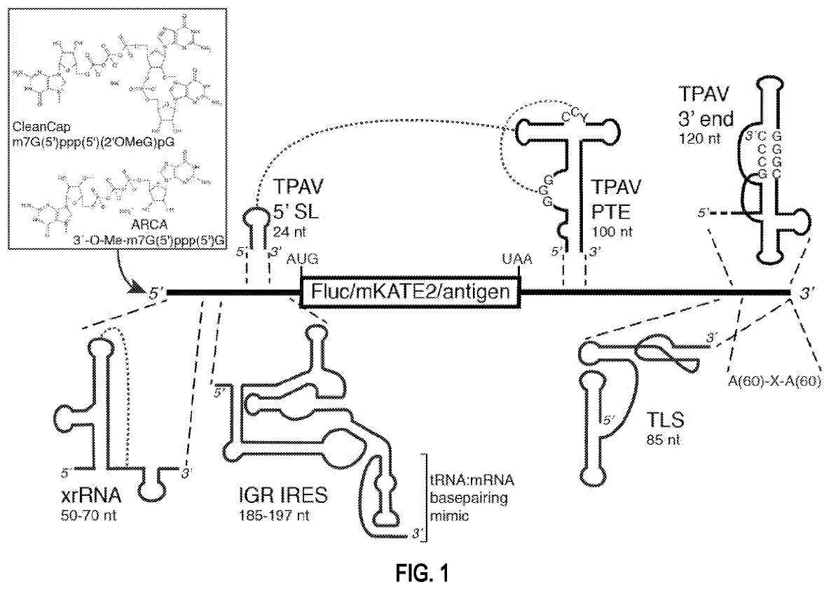

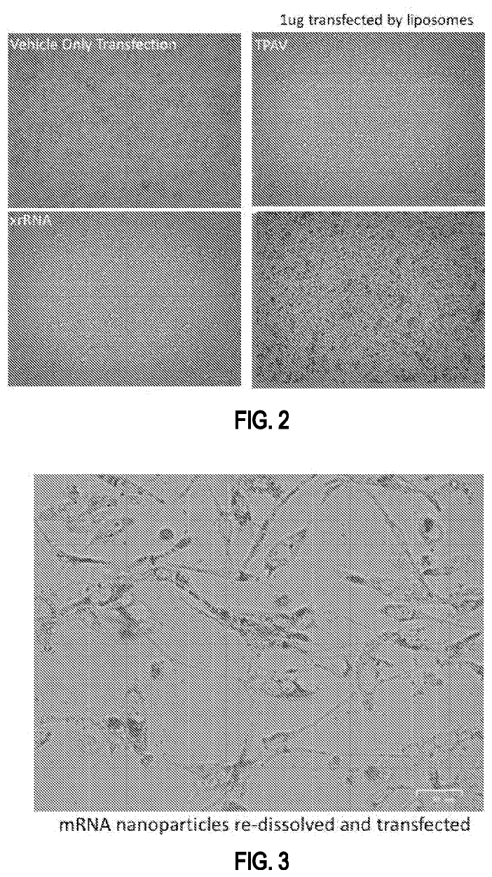

Diagrams of exemplary expression cassettes are shown in FIG. 1. FIG. 2 shows accumulation of the mKATE fluorescent reporter protein after transfection with mRNA containing the indicated UTRs.

### Example 2: Thermostable Polyanhydride Nanoparticles

Incorporation of mRNA into Polyanhydride Nanoparticles

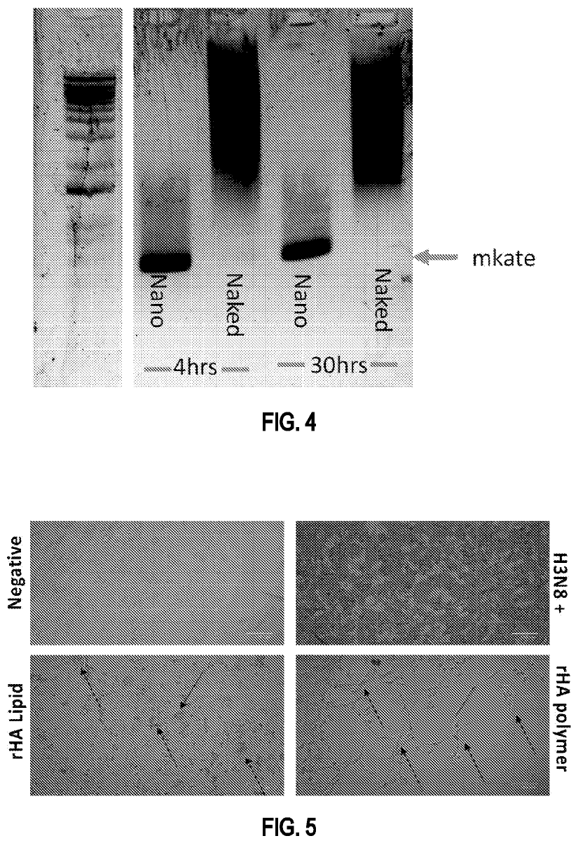

mRNA was successfully incorporated into polyanhydride nanoparticles (based on 20:80 CPTEG:CPH formulations) using an xrRNA expression cassette. CPTEG specifically is 1,8-bis-(p-carboxyphenoxy)-3,6-dioxaoctane while CPH is 1,6-bis-(p-carboxyphenoxy)-hexane which can be blended in different ratios to change release kinetics as well as protection from water and oxygen. Specifically, 800 μg of mRNA was generated by traditional IVT expression using Lucigen T7 mega kit. The RNA was purified by phenol/chloroform extraction, salt and glycogen carrier. The RNA pellet was resuspended in pure water with 0.1% spermine (Sigma). The RNA was then freeze dried and encapsulated into 20:80 CPTEG/CPH. The mRNA-containing nanoparticles (5 mg) were then dissolved into water, subjected the RNA to reverse transcription and then PCRed using mKATE primers and found the product. Some of the RNA was also blended with lipofectamine and placed on A549 cells and fluorescent protein was found in the cells 24 hours later. The same approach was repeated but omitting the nanoparticle at room temperature and a similar finding occurred. FIG. 3 shows that mRNA extracted from nanoparticles can still transfect cells. FIG. 4 shows that mRNA extracted from nanoparticles are likely protected from thermodegradation at room temperature. For comparisons, both the Moderna and Pfizer/BioNtech vaccine mRNAs have a room temperature shelf life of approximately four hours before breaking down.

mRNA Transfection in Copolymers Leads to Higher Transfection Efficiencies than Lipid Transfections

Whether the copolymer could transfect cells on par with lipids designed to deliver mRNA optimally to cells was tested. The copolymer was capable of delivery into the cells with enhanced delivery of equine HA mRNA over lipid (FIG. 5).

mRNA in Polyanhydride Carriers are Thermostable and can Transfect Months after being Stored on the Shelf

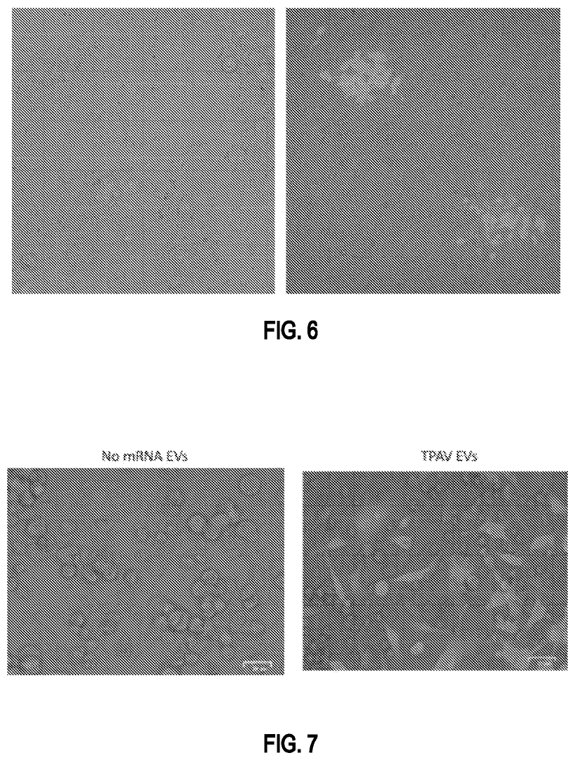

Formulations of 40 μg of mRNA coding for RSV F protein were left on the shelf for 4 months, one in polyanhydride 20:80 and one without. Only the one in polyanhydride was stable (FIG. 6). Higher transfection efficiencies could be obtained but getting mRNA back out of 20:80 quickly can be difficult as it is designed to dissolve much more slowly. Similar studies were done at 37° C. with mRNA and RNA viruses and were found to be stable only in polyanhydride material.

### Example 3: Extracellular Vesicles

The TPAV/mKATE expression cassette was PCR amplified off a commercially synthesized plasmid by overlapping oligos. The PCR product includes a T7 promoter (5′-TAATACGACTCACTATAG-3′) in front of the 5′UTR. The PCR product (5 μg) was transfected by DEA Dextran and 10% glycerol shock into A549 cells that were previously engineered to express T7 polymerase continuously (A549-T7 cells). Transfection reagents were rinsed and replaced with standard media but without serum after 2 hours. Three days later, the supernatant was harvested, centrifuged at 3000 g and filtered with 0.2 μm filters twice and 100 μl of the 40 ml was added back onto a 6 well plate containing HELA cells with no T7 polymerase. The cells were observed for 4 days and mKATE fluorescence was observed after 2 days (FIG. 7). Brightness of red fluorescence and number of fluorescent cells increased over the entire time period.

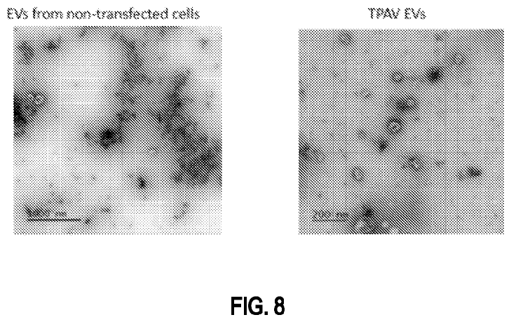

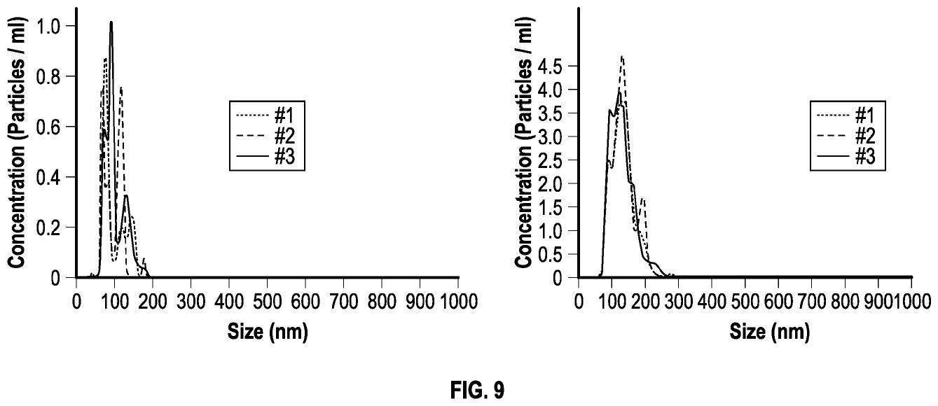

The EVs were spun down using high speed ultracentrifugation and the final product was diluted in 100 μl of water. They then diluted it 500× and imaged by scanning electron microscopy. In tandem, the EVs were imaged by a Nanosight particle imager (FIG. 8). mKATE mRNA was detected from EVs harvested by a commercial kit suggesting the transcripts are being incorporated and protected from degradation for the week before the EVs were harvested from 4° C.-stored pre-clarified cell culture supernatant (FIG. 9).

### Example 4: RNA Protein Binding Domain

Additional TPAV and xrRNA systems were developed that incorporate an RNA protein binding domain (SEQ ID NO: 11) that targets to either lipid membranes or MVP protein. These will enhance the incorporation of the mRNA into exosomes. Inclusion of a T7 stop sequence at the end of the expression cassette ensures that an uncut DNA plasmid can be transfected into cells for transcription rather than needing to cut and transfect linear plasmids. The use of the MVP protein binding domain also allows for capture of the RNA using a recombinant version of the protein immobilized onto column for capture of the RNA after lysis of cells that have undergone transcription through the T7 polymerase.

### Example 5: Dual Expression System

A dual expression system was developed that incorporates an IRES sequence from polio virus and another IRES from the cricket paralysis virus. This system allows for dual expression of two proteins. Expression of two proteins for a vaccine are possible. Inclusion of heavy and light chain antibodies sequences with a multiple cloning site also allows for antibodies to be cloned by the CDR3 region and ligated in frame with the antibodies (one for light chain and one for heavy chain). This then allows for the dual expression of the full heavy and light chain within targeted cells in a vaccinated animal/human for generation of therapeutic antibodies to be made and secreted in the body.

A system was also developed that uses the cricket paralysis virus IRES for downstream generation of heavy chain antibodies and an upstream using the TPAV/xrRNA system for the light chain of antibodies. The goal is to use this mRNA for making antibodies in vivo for therapeutics.

### Example 6: Additional Expression Systems

A number of new expression systems were developed for testing. The first system uses a cricket paralysis virus IRES (SEQ ID NO: 18) in conjunction with the other codes. The second uses a Triticum mosaic virus (TRIMV) IRES (SEQ ID NO: 19) in conjunction with the other codes. Two additional systems were developed that use either both or a single UTR from tomato bushy stunt virus (SEQ ID NO: 20 and 21).

### Example 7: Self-Cleaving Cassettes

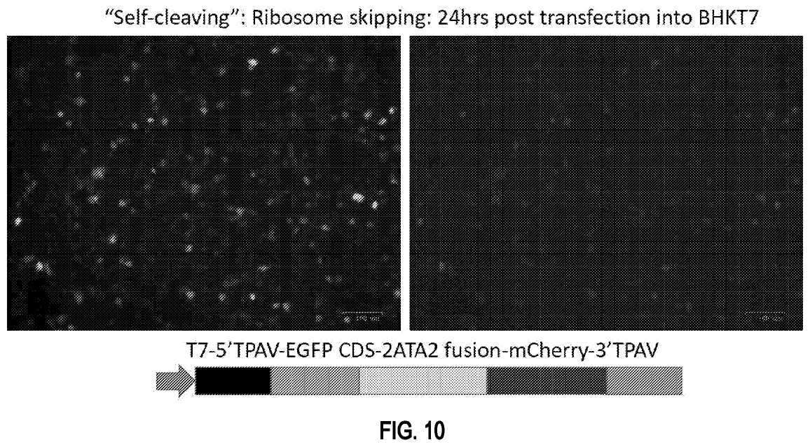

A polycistronic system using self-cleaving peptides was tested. The use of the dual 2A, TA2 peptides placed between two coding sequences on the mRNA constructs allowed for a near 1:1 expression level of both proteins (FIG. 10). This is unusual as the traditional use of an IRES between coding sequences often leads to poor expression of the downstream gene. Thus, the mRNA constructs can be designed to make one, two, or more proteins off the same mRNA construct. The dual peptides ensure more of the proteins are separate proteins rather than fusions. This system is an option to express more than one flu protein within the same APC and reduce manufacturing costs versus multiple mRNA constructs.

### Example 8: Comparison of Additional mRNA Constructs

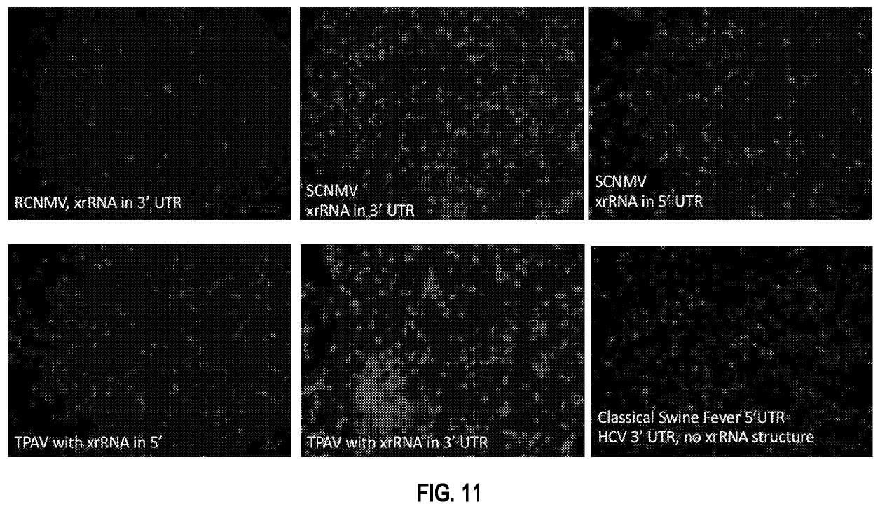

Additional mRNA constructs were expressed in T7 BHK cells. Expression levels from the various constructs showed high levels of expression of the reporter protein. Among those tested, SCNMV and TPAV with 3′ xrRNA gave the most favorable protein expression of mCherry (FIG. 11) and mRNA for incorporation into EVs (not shown). These data demonstrate an ability to tailor the individual UTRs to the gene of interest to ensure high level of expression.

The mRNA constructs have 10 bp leaders before the xrRNA in the 5′ UTR and thus the lower expression shown could be from xrRNA getting access to the transcript rather than being blocked with a xrRNA that starts right at the 5′ end. Future studies will optimize the 5′ xrRNA for comparison to the 3′ xrRNA UTRs.

Additional mRNA constructs are summarized in Table 1.

TABLE 1

| 5′ UTR | 3′ UTR |
|---|---|
| Classical swine fever virus | Hepatitis C virus |
| (SEQ ID NO: 29) | (SEQ ID NO: 30) |
| RCNMV (SEQ ID NO: 37) | TPAV (SEQ ID NO: 8) |
| SCNMV (SEQ ID NO: 28) | TPAV (SEQ ID NO: 8) |
| Cricket paralysis virus IRES | TPAV (SEQ ID NO: 8) |
| (SEQ ID NO: 10) | |
| Zika virus xrRNA (SEQ ID NO: 6) | TPAV (SEQ ID NO: 8) |
| TBSV (SEQ ID NO: 14) | TPAV (SEQ ID NO: 8) |
| TRIMV IRES (SEQ ID NO: 12) | TPAV (SEQ ID NO: 8) |
| CHOP IRES (SEQ ID NO: 34) | Zika virus (SEQ ID NO: 35) |
| Bovine viral | Potato leafroll virus xrRNA |
| diarrhea virus | (SEQ ID NO: 31), |
| (SEQ ID NO: 32) | TPAV (SEQ ID NO: 8) |
| Opium poppy mosaic virus | TPAV (SEQ ID NO: 8) |
| (SEQ ID NO: 33) | |

### Example 9: mRNA Vaccination

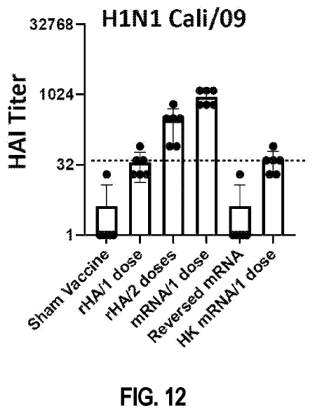

Mice were vaccinated with rHA from H3N8 once or twice with Alum or with mRNA on the TPAV cassette for H3N8 HA in the correct or reversed orientation in EVs or β-actin (UTR) cap and tailed H3N8 HA in liposomes. Hemagglutination inhibition (HAI) was assessed (FIG. 12). The mRNA vaccines outperformed the Flu HA protein in eliciting neutralizing antibodies. These data strongly indicate that the mRNA constructs could lead to protection from flu challenge.

### Example 10: Naked mRNA Transfection

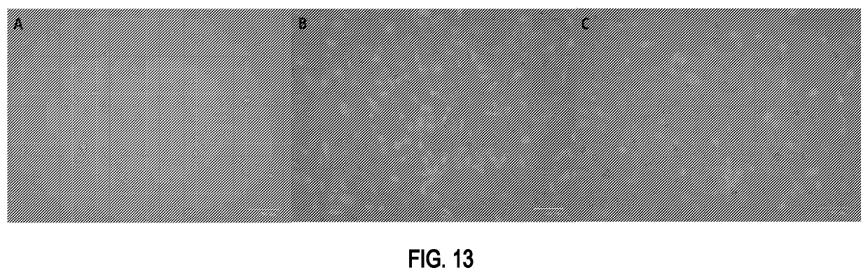

A549 cells without T7 were transfected with sham, a swine cassette expressing mCherry, or commercial mRNA expressing mCherry (modified U/C, Arco cap, and tailed) using Ribojuice liposomes. Actual expression of mRNA compared to the commercial mRNA using modified bases and 5′ caps and poly(A) tails (FIG. 13). These data suggest that the mRNA constructs of the disclosure should express their genes with high levels that should then evoke an immune response when injected into animals.

### Example 11: mRNA Stability in Polyanhydride Nanoparticles

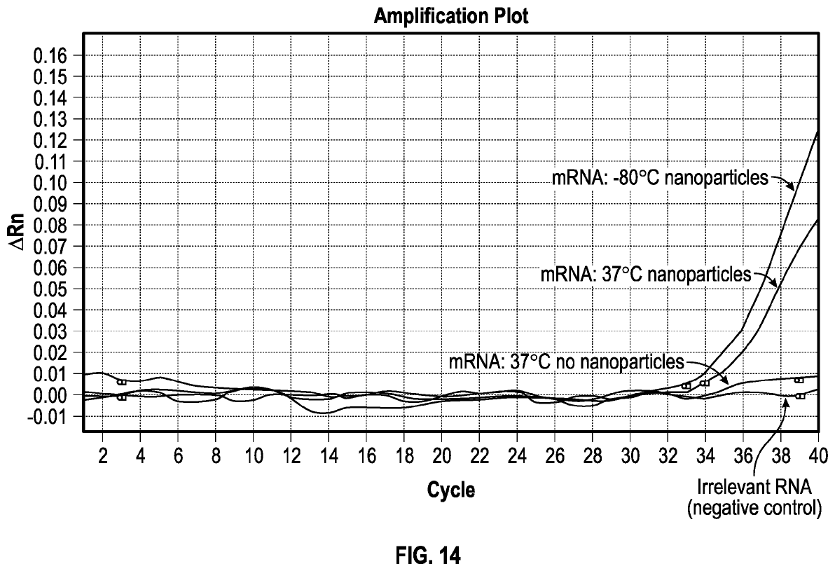

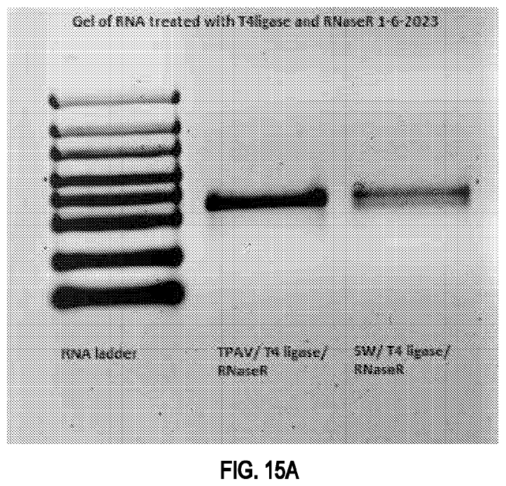

A thermostability assay demonstrated that the mRNA constructs placed in polyanhydrides exhibit resistance to degradation from heat after 7 days at 37° C. (FIG. 14). Traditional IVT generated mRNA did not have this level of thermostability. Thus, the mRNA should be room temperature stable when placed within the polyanhydride nanoparticles or rods.

### Example 12: Circular RNA Construction

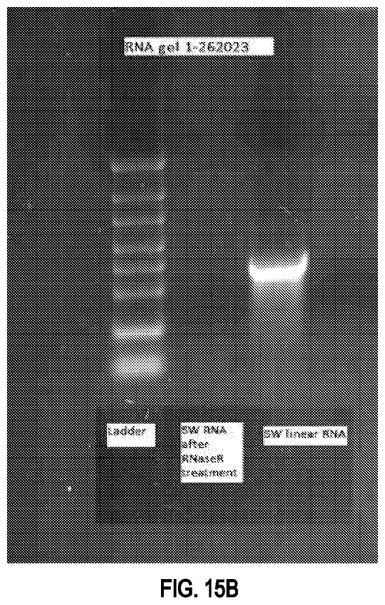

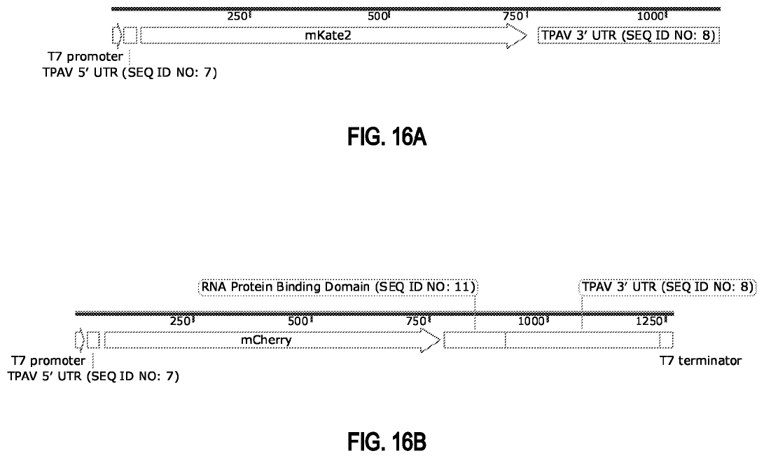

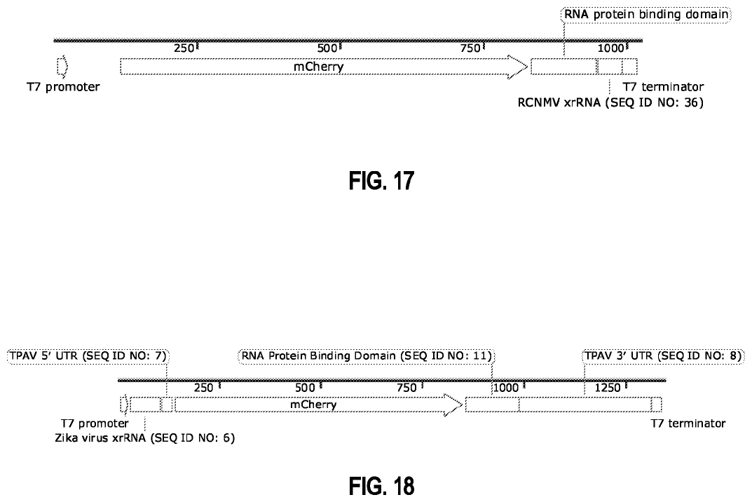

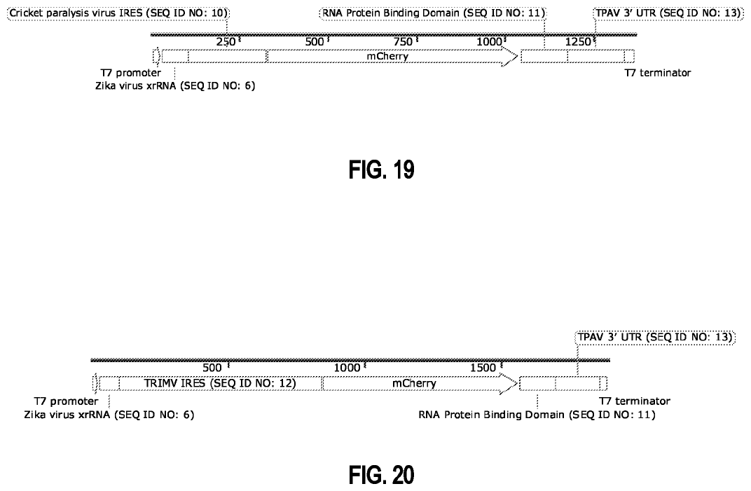

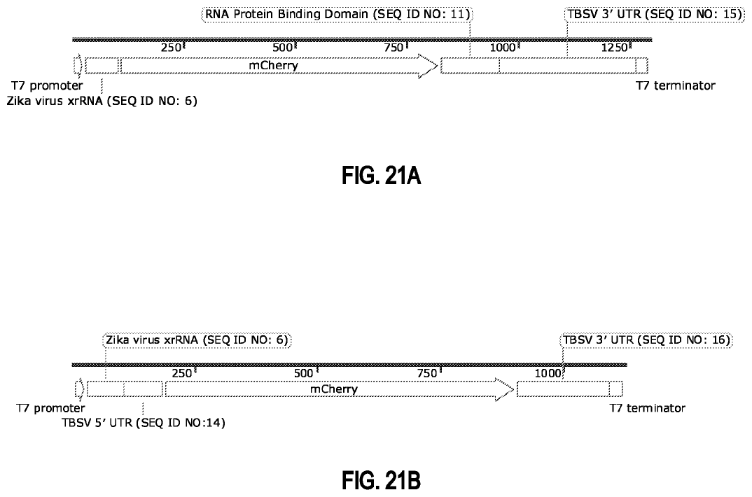

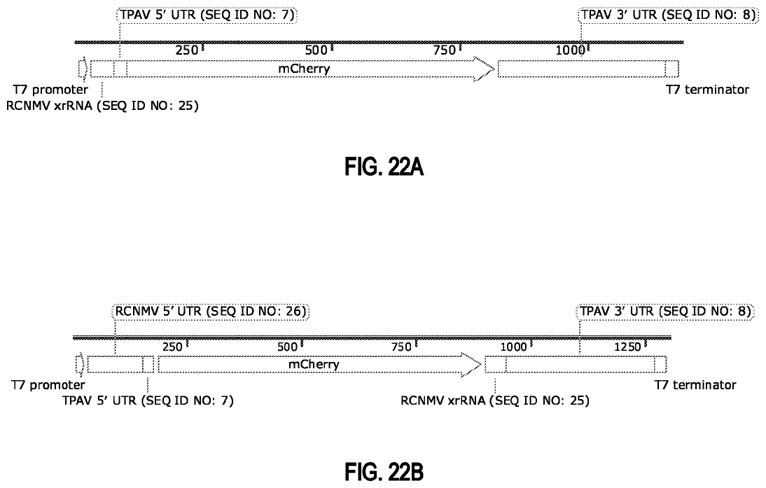

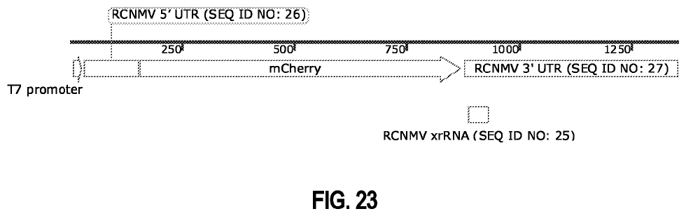

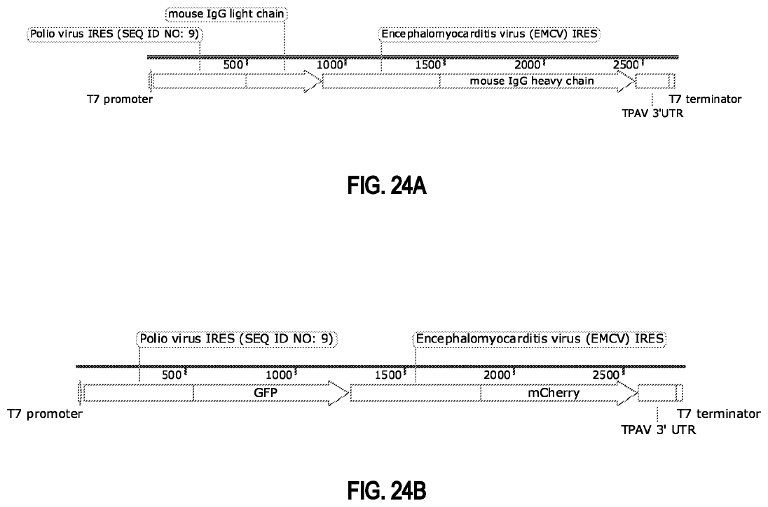

Through removal of the 5′ triphosphate and addition of a single phosphate group with a kinase, the mRNA constructs of the disclosure can be circularized with RNA ligase 1 (FIG. 15). Using circular RNA further alleviates the need to include modified bases and also allows for digestion of dsRNA and short RNA products by using T4 RNA ligase 1, which only ligates linear ssRNA (not dsRNA as with T4 RNA ligase 2). This could allow skipping steps in the manufacture process to remove dsRNA and short products.

The invention claimed is:

1. An RNA polynucleotide comprising a 5′ untranslated region (5′ UTR), a heterologous sequence encoding at least one polypeptide, and a 3′ untranslated region (3′ UTR), wherein the 5′ UTR or the 3′ UTR comprise a panicum mosaic virus-like cap independent translation enhancer or an exoribonuclease-resistant RNA (xrRNA) element.

2. The RNA polynucleotide of claim 1, wherein the 5′ UTR or the 3′ UTR comprises an xrRNA element.

3. The RNA polynucleotide of claim 1, wherein the xrRNA element comprises SEQ ID NO: 6, 25, 31, or 36.

4. The RNA polynucleotide of claim 1, wherein the 5′ UTR or the 3′ UTR comprises a panicum mosaic virus-like cap independent translation enhancer.

5. The RNA polynucleotide of claim 1, wherein the 5′ UTR or the 3′ UTR is from Thin paspalum asymptomatic virus (TPAV), Tomato Bushy Stunt Virus (TBSV), Sweet clover necrotic mosaic virus (SCNMV), Red clover necrotic mosaic virus (RCNMV), or Opium poppy mosaic virus (OPMV).

6. The RNA polynucleotide of claim 1, wherein the 5′ UTR or the 3′ UTR comprises a polynucleotide that has at least 80%, at least 90%, at least 95%, at least 96%, at least 97%, at least 98%, at least 99%, or 100% sequence identity to SEQ ID NO: 6, 7, 8, 9, 10, 11, 12, 13, 14, 15, 16, 25, 26, 27, 28, 29, 30, 31, 32, 33, 34, 35, 36, 37, or 38.

7. The RNA polynucleotide of claim 1, wherein the 5′ UTR comprises SEQ ID NO: 7, 14, 26, 28, 29, 32, 33, or 37, and wherein the 3′ UTR comprises SEQ ID NO: 8, 13, 15, 16, 27, 30, 35, or 38.

8. The RNA polynucleotide of claim 1, wherein the RNA polynucleotide comprises an internal ribosome entry site (IRES).

9. The RNA polynucleotide of claim 1, wherein IRES comprises SEQ ID NO: 9, 10, 12, or 34.

10. The RNA polynucleotide of claim 1, wherein the RNA polynucleotide comprises an RNA protein binding domain.

11. The RNA polynucleotide of claim 1, wherein the RNA polynucleotide does not comprise a 5′ cap structure or a poly-A tail.

12. The RNA polynucleotide of claim 1, wherein the RNA polynucleotide does not comprise a modified nucleoside.

13. The RNA polynucleotide of claim 1, wherein the at least one polypeptide comprises an antigenic polypeptide.

14. The RNA polynucleotide of claim 13, wherein the antigenic polypeptide is from an influenza virus or a coronavirus.

15. The RNA polynucleotide of claim 1, wherein the at least one polypeptide comprises a light chain and a heavy chain of an antibody.

16. A polyanhydride composition comprising a polyanhydride polymer and the RNA polynucleotide of claim 1, wherein the polyanhydride polymer encapsulates the RNA polynucleotide.

17. The composition of claim 16, wherein the polyanhydride polymer comprises copolymers of 1,8-bis(carboxyphenoxy)-3,6-dioxaoctane (CPTEG) and 1,6-bis-(p-carboxyphenoxy) hexane (CPH).

18. The composition of claim 17, wherein the CPTEG:CPH ratio is about 20:80 to about 50:50.

19. An exosome for delivery of a RNA polynucleotide, the exosome comprising the RNA polynucleotide of claim 1.

20. A pharmaceutical composition comprising:
the RNA polynucleotide of claim 1; and
a pharmaceutically acceptable excipient.

21. A method of producing a polypeptide of interest in a subject, delivering an RNA polynucleotide to a subject, inducing an immune response in a subject, or delivering an antibody to a subject, the method comprising:
administering to the subject the RNA polynucleotide of claim 1, or a polyanhydride composition, an exosome, or a pharmaceutical composition comprising the RNA polynucleotide.

22. The method of claim 21, wherein the RNA polynucleotide, the polyanhydride composition, the exosome, or the pharmaceutical composition is administered to the subject intramuscularly, intranasally, or intradermally.

23. The method of claim 21, wherein the subject is a human.

24. A method for producing exosomes for delivery of an RNA polynucleotide, the method comprising:
transforming a cell with a polynucleotide construct that expresses the RNA polynucleotide of claim 1;
culturing the cell in a growth media, wherein exosomes comprising the RNA polynucleotide are released into the extracellular growth media;
removing the cells from the growth media; and
harvesting the exosomes comprising the RNA polynucleotide from the growth media.

25. The method of claim 24, wherein the harvesting is by ultracentrifugation.

26. The method of claim 25, wherein the cell is an A549 cell.

27. The method of claim 24, wherein the polynucleotide construct comprises a promoter.


SEQUENCE LISTING (38 sequences)

SEQ ID NO: 1 - RNA, 1094 nt, synthetic construct
  source 1..1094: mol_type = other RNA
ctaatacgactcactatagggtattggctgcaacctatacctcacgggatccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgatctagaaatctagatagactcgctcagagtcatcctaggctaggaccggccagacaaaggtccgacactacatgcagcgctctaggtgtgtagtgacagtaagcgcgctctatccgaaactccagtgacaggacaagaccccaggcgggacgccacacccatgtttgcagacatggcccgaggaaactctggtgtgggctggggggaatcctatgatcactggacggaaggtagagagttccagcacaacccctttagggtgggccgtaagtgcgtgggaacatttcctacagtctcactgagaagattggacggggtaccatgagtacccctcaccaggccc

SEQ ID NO: 2 - RNA, 1262 nt, synthetic construct
  source 1..1262: mol_type = other RNA
ctaatacgactcactatagggataattattggctgcaacctatacctcacgggatccgccgccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggggacgacgatgacacgatactttgtcggccgaactcgctgtttaactgcccggcgagatcgcagggtgttgtgctattcgcgtgccgtgtgcatacgccgatcacatgaccatctagaaatctagatagactcgctcagagtcatcctaggctaggaccggccagacaaaggtccgacactacatgcagcgctctaggtgtgtagtgacagtaagcgcgctctatccgaaactccagtgacaggacaagaccccaggcgggacgccacacccatgtttgcagacatggcccgaggaaactctggtgtgggctggggggaatcctatgatcactggacggaaggtagagagttccagcacaacccctttagggtgggccgtaagtgcgtgggaacatttcctacagtctcactgagaagattggacggggtaccatgagtacccctcaccaggcccatccatgatatctgttagtttttttc

SEQ ID NO: 3 - RNA, 1023 nt, synthetic construct
  source 1..1023: mol_type = other RNA
aagcttctaatacgactcactatagggataattaaacctcgatcataaagactaattgattccaagcaagcaaagactgtaagactacgaacaatataggaaggtttgagggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggggacgacgatgacacgatactttgtcggccgaactcgctgtttaactgcccggcgagatcgcagggtgttgtgctattcgcgtgccgtgtgcatacgccgatcacatgaccagggcgtaacctccatccgagttgcaagagagggaaacgcagtctcatccatgatatctgttagtttttttccatatg

SEQ ID NO: 4 - RNA, 2674 nt, synthetic construct
  source 1..2674: mol_type = other RNA
aagcttctaatacgactcactatagggataattaaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaaccggcgaggcgcgccaagtttaaacaccatggaagcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagaattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaaagctagcagcgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctgatgattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccgcaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaataaaactcgaggggacgacgatgacacgatactttgtcggccgaactcgctgtttaactgcccggcgagatcgcagggtgttgtgctattcgcgtgccgtgtgcatacgccgatcacatgaccagggcgtaacctccatccgagttgcaagagagggaaacgcagtctcatccatgatatctgttagtttttttccatatgggtacc

SEQ ID NO: 5 - RNA, 2775 nt, synthetic construct
  source 1..2775: mol_type = other RNA
aagcttctaatacgactcactatagggataattaaagttcaatagaagggggtacaaaccagtaccaccacgaacaagcacttctgtttccccggtgctgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgttgagtctggacatccctcaccggtgacggtggtccaggctgcgttggcggcctacctatggctaacgccatgggacgctagttgtgaacaaggtgtgaagagcctattgagctacataagaatcctccggcccctgagtgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaagatctatgtccaagggcgaggaactgttcacgggcgtcgtcccgatcctcgtcgaactcgatggcgacgtcaacggccacaagttctccgtcagcggtgaaggtgaaggcgacgccacctacggcaagctcacccttaagttcatctgcaccaccggcaaactgccggtgccgtggccgacgctggtcacgaccctcacgtatggcgttcagtgcttctcgcgctacccggaccacatgaagcagcacgatttcttcaagtcggcgatgccggaaggctacgtccaggaacgcacgatcttcttcaaggacgatggcaactacaagacccgcgccgaagtcaagttcgagggcgacacgcttgtgaatcgcatcgaacttaagggcatcgacttcaaggaagatggcaacatcctcggccacaagctggagtataactacaattcgcacaacgtctacatcatggctgacaagcagaagaatggtatcaaggttaacttcaagatccgccacaacatcgaagacggctccgtccagctggcggaccactatcagcagaacaccccgatcggcgacggcccggttctcctcccggataaccactacctcagcacgcagtcggcgctgtcgaaggacccgaatgaaaagcgcgaccacatggtccttctggagttcgtgaccgcggcgggcatcacgcacggcatggacgaactttacaagtaagaattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgtctagagtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacgtttaaacagtacgaacgcgccgagggccgccactccaccggcggcatggacgagctgcaagtagcggccgcactcgaggggacgacgatgacacgatactttgtcggccgaactcgctgtttaactgcccggcgagatcgcagggtgttgtgctattcgcgtgccgtgtgcatacgccgatcacatgaccagggcgtaacctccatccgagttgcaagagagggaaacgcagtctcatccatgatatctgttagtttttttccatatg

SEQ ID NO: 6 - RNA, 73 nt, Zika virus
  source 1..73: mol_type = genomic RNA
taaggtcaggccggcgaaagtcgccacagtttggggaaagctgtgcagcctgtaacccccccacgaaagtggg

SEQ ID NO: 7 - RNA, 25 nt, Thin paspalum asymptomatic virus
  source 1..25: mol_type = genomic RNA
tattggctgcaacctatacctcacg

SEQ ID NO: 8 - RNA, 325 nt, Thin paspalum asymptomatic virus
  source 1..325: mol_type = genomic RNA
ctcgctcagagtcatcctaggctaggaccggccagacaaaggtccgacactacatgcagcgctctaggtgtgtagtgacagtaagcgcgctctatccgaaactccagtgacaggacaagaccccaggcgggacgccacacccatgtttgcagacatggcccgaggaaactctggtgtgggctggggggaatcctatgatcactggacggaaggtagagagttccagcacaacccctttagggtgggccgtaagtgcgtgggaacatttcctacagtctcactgagaagattggacggggtaccatgagtacccctcaccaggccc

SEQ ID NO: 9 - RNA, 502 nt, Enterovirus C
  source 1..502: mol_type = genomic RNA
gttcaatagaagggggtacaaaccagtaccaccacgaacaagcacttctgtttccccggtgctgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgttgagtctggacatccctcaccggtgacggtggtccaggctgcgttggcggcctacctatggctaacgccatgggacgctagttgtgaacaaggtgtgaagagcctattgagctacataagaatcctccggcccctgagtgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaagatct

SEQ ID NO: 10 - RNA, 219 nt, Cricket paralysis virus
  source 1..219: mol_type = genomic RNA
taattctacacccggaattcaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctgaag

SEQ ID NO: 11 - RNA, 131 nt, synthetic construct
  source 1..131: mol_type = other RNA
gggacgacgatgacacgatactttgtcggccgaactcgctgtttaactgcccggcgagatcgcagggtgttgtgctattcgcgtgccgtgtgcatacgccgatcacatgaccatctagaaatctagataga

SEQ ID NO: 12 - RNA, 738 nt, Triticum mosaic virus
  source 1..738: mol_type = genomic RNA
aaattaaagatcatattacataaaataacataatataaaatcacttaaaatcatgtgttttaaactacgcttagtttaattagttttggtgcgtttagcgattcgtcattgtacatggtgtgttgtgtgttttatgattttagtatgtttcttaaattattgaagccctataaggaccggctataaacgtcctgttttcaagtgggaaaagaaaccactcgccttaccactagctgggatctagctagagctccggcgtaaaacgagctacgcttttggatgcagcgttacgcattcctgggcttaggcgattgtactacaatgggtagcccccagtgccagtttctggcccgctattgtattacaattcggttaagttaacttggttggaaacaagccaaatgctagctatcattcgcattcggacatgaggaaggtgaacgcagtgaatcatagtggtggtacgctcttggggtggttcccaagacttcgtagggctatggttagctgttagtaagacctaatgttcgtttgtgatacagtcgaaagttgtttccgtatggagctcggtctgcgcgttaagcaccagcctgactatgggcagtatccctgtttttccactattctcactatcaaacacaacgcacgactttctgctctcttggcactttcttactttcacactctcgcgctcgtttcaaagttttattacttctctttttctcctgaccattcacg

SEQ ID NO: 13 - RNA, 161 nt, Thin paspalum asymptomatic virus
  source 1..161: mol_type = genomic RNA
ctctggtgtgggctggggggaatcctatgtatgatcactggacggaaggtagagagttccagcacaacccctttagggtgggccgtaagtgcgtgggaacatttcctacagtctcactgagaagattggacggggtaccatgagtacccctcaccaggccc

SEQ ID NO: 14 - RNA, 78 nt, Tomato bushy stunt virus
  source 1..78: mol_type = genomic RNA
agaaattctccaggatttctcgacctagttcgtttatctggtgacttgcgctaccgttgctttgcgtagagaatttct

SEQ ID NO: 15 - RNA, 306 nt, Tomato bushy stunt virus
  source 1..306: mol_type = genomic RNA
gttcgcggaaacgagtgtaaatctgcatagcatacaggttacctccttgttgggttctagatgttatgatgacgagtcggtcggctccgcgctaggtttggtcgcctaggggatggagatatggaaagggtctcgtgtgctgtcagtcggtcgaaagacgcgcttgcaacatgggcctataaccggataagtcatagcaatactagccaacatgaattggattcctgtttacgaaagttaggtgacacttgtggaaacggacctagacacggttgatctcacttcgggggggctatagagatcgct

SEQ ID NO: 16 - RNA, 187 nt, Tomato bushy stunt virus
  source 1..187: mol_type = genomic RNA
gggacgacgatgacacgatactttgtcggccgaatttggatgttaggatgacgagtcgactgcgggctccgcactaggtttggtcgcctagaggatggagatatggaaagggtctcgtgtggtatcagtcggtcgaaagacgcgcttccaacatgggcctatggtcggataagtcttagcaatacca

SEQ ID NO: 17 - RNA, 1344 nt, synthetic construct
  source 1..1344: mol_type = other RNA
aagcttctaatacgactcactatagggataattaaggtcaggccggcgaaagtcgccacagtttggggaaagctgtgcagcctgtaacccccccacgaaagtggggggtattggctgcaacctatacctcacgatgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggggacgacgatgacacgatactttgtcggccgaactcgctgtttaactgcccggcgagatcgcagggtgttgtgctattcgcgtgccgtgtgcatacgccgatcacatgaccatctagaaatctagatagactcgctcagagtcatcctaggctaggaccggccagacaaaggtccgacactacatgcagcgctctaggtgtgtagtgacagtaagcgcgctctatccgaaactccagtgacaggacaagaccccaggcgggacgccacacccatgtttgcagacatggcccgaggaaactctggtgtgggctggggggaatcctatgatcactggacggaaggtagagagttccagcacaacccctttagggtgggccgtaagtgcgtgggaacatttcctacagtctcactgagaagattggacggggtaccatgagtacccctcaccaggcccatccatgatatctgttagtttttttccatatg

SEQ ID NO: 18 - RNA, 1367 nt, synthetic construct
  source 1..1367: mol_type = other RNA
aagcttctaatacgactcactatagggataattaaggtcaggccggcgaaagtcgccacagtttggggaaagctgtgcagcctgtaacccccccacgaaagtgggtaattctacacccggaattcaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctgaagggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggggacgacgatgacacgatactttgtcggccgaactcgctgtttaactgcccggcgagatcgcagggtgttgtgctattcgcgtgccgtgtgcatacgccgatcacatgaccatctagaaatctagatagactctggtgtgggctggggggaatcctatgtatgatcactggacggaaggtagagagttccagcacaacccctttagggtgggccgtaagtgcgtgggaacatttcctacagtctcactgagaagattggacggggtaccatgagtacccctcaccaggcccatccatgatatctgttagtttttttcatatg

SEQ ID NO: 19 - RNA, 1889 nt, synthetic construct
  source 1..1889: mol_type = other RNA
aagctttaatacgactcactatagggataattaaggtcaggccggcgaaagtcgccacagtttggggaaagctgtgcagcctgtaacccccccacgaaagtgggaaattaaagatcatattacataaaataacataatataaaatcacttaaaatcatgtgttttaaactacgcttagtttaattagttttggtgcgtttagcgattcgtcattgtacatggtgtgttgtgtgttttatgattttagtatgtttcttaaattattgaagccctataaggaccggctataaacgtcctgttttcaagtgggaaaagaaaccactcgccttaccactagctgggatctagctagagctccggcgtaaaacgagctacgcttttggatgcagcgttacgcattcctgggcttaggcgattgtactacaatgggtagcccccagtgccagtttctggcccgctattgtattacaattcggttaagttaacttggttggaaacaagccaaatgctagctatcattcgcattcggacatgaggaaggtgaacgcagtgaatcatagtggtggtacgctcttggggtggttcccaagacttcgtagggctatggttagctgttagtaagacctaatgttcgtttgtgatacagtcgaaagttgtttccgtatggagctcggtctgcgcgttaagcaccagcctgactatgggcagtatccctgtttttccactattctcactatcaaacacaacgcacgactttctgctctcttggcactttcttactttcacactctcgcgctcgtttcaaagttttattacttctctttttctcctgaccattcacgggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggggacgacgatgacacgatactttgtcggccgaactcgctgtttaactgcccggcgagatcgcagggtgttgtgctattcgcgtgccgtgtgcatacgccgatcacatgaccatctagaaatctagatagactctggtgtgggctggggggaatcctatgtatgatcactggacggaaggtagagagttccagcacaacccctttagggtgggccgtaagtgcgtgggaacatttcctacagtctcactgagaagattggacggggtaccatgagtacccctcaccaggcccatccatgatatctgttagtttttttccatatg

SEQ ID NO: 20 - RNA, 1295 nt, synthetic construct
  source 1..1295: mol_type = other RNA
aagcttaatacgactcactatagggataattaaggtcaggccggcgaaagtcgccacagtttggggaaagctgtgcagcctgtaacccccccacgaaagtgggggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggggacgacgatgacacgatactttgtcggccgaactcgctgtttaactgcccggcgagatcgcagggtgttgtgctattcgcgtgccgtgtgcatacgccgatcacatgaccatctagaaatctagatagagttcgcggaaacgagtgtaaatctgcatagcatacaggttacctccttgttgggttctagatgttatgatgacgagtcggtcggctccgcgctaggtttggtcgcctaggggatggagatatggaaagggtctcgtgtgctgtcagtcggtcgaaagacgcgcttgcaacatgggcctataaccggataagtcatagcaatactagccaacatgaattggattcctgtttacgaaagttaggtgacacttgtggaaacggacctagacacggttgatctcacttcgggggggctatagagatcgctatccatgatatctgttagtttttttccatatg

SEQ ID NO: 21 - RNA, 1125 nt, synthetic construct
  source 1..1125: mol_type = other RNA
aagcttctaatacgactcactatagggataattaaggtcaggccggcgaaagtcgccacagtttggggaaagctgtgcagcctgtaacccccccacgaaagtgggagaaattctccaggatttctcgacctagttcgtttatctggtgacttgcgctaccgttgctttgcgtagagaatttctatgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggggacgacgatgacacgatactttgtcggccgaatttggatgttaggatgacgagtcgactgcgggctccgcactaggtttggtcgcctagaggatggagatatggaaagggtctcgtgtggtatcagtcggtcgaaagacgcgcttccaacatgggcctatggtcggataagtcttagcaataccaatccatgatatctgttagtttttttccatatg

SEQ ID NO: 22 - RNA, 1181 nt, synthetic construct
  source 1..1181: mol_type = other RNA
aagcttctaatacgactcactatagggataatgcgtaacctccatccgagttgcaagagagggaaacgcagtctcgtattggctgcaacctatacctcacgatgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagctcgctcagagtcatcctaggctaggaccggccagacaaaggtccgacactacatgcagcgctctaggtgtgtagtgacagtaagcgcgctctatccgaaactccagtgacaggacaagaccccaggcgggacgccacacccatgtttgcagacatggcccgaggaaactctggtgtgggctggggggaatcctatgatcactggacggaaggtagagagttccagcacaacccctttagggtgggccgtaagtgcgtgggaacatttcctacagtctcactgagaagattggacggggtaccatgagtacccctcaccaggcccatccatgatatctgttagtttttttccatatg

SEQ ID NO: 23 - RNA, 1301 nt, synthetic construct
  source 1..1301: mol_type = other RNA
aagcttctaatacgactcactatagggataatgaacaaacgttttaccggtttgtaaattggaattctcgaattcgccagtcgccttcttcgtatcaaggatctggaatttgaacggtatctttatcattttaatttctacttttcgtaccatattggctgcaacctatacctcacgatgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggcgtaacctccatccgagttgcaagagagggaaacgcagtctcgctcgctcagagtcatcctaggctaggaccggccagacaaaggtccgacactacatgcagcgctctaggtgtgtagtgacagtaagcgcgctctatccgaaactccagtgacaggacaagaccccaggcgggacgccacacccatgtttgcagacatggcccgaggaaactctggtgtgggctggggggaatcctatgatcactggacggaaggtagagagttccagcacaacccctttagggtgggccgtaagtgcgtgggaacatttcctacagtctcactgagaagattggacggggtaccatgagtacccctcaccaggcccatccatgatatctgttagtttttttccatatg

SEQ ID NO: 24 - RNA, 1351 nt, synthetic construct
  source 1..1351: mol_type = other RNA
aagcttctaatacgactcactatagggataatgaacaaacgttttaccggtttgtaaattggaattctcgaattcgccagtcgccttcttcgtatcaaggatctggaatttgaacggtatctttatcattttaatttctacttttcgtaccagccatgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagaatgccctagcgtaacctccatccgagttgcaagagagggaaacgcagtctcgttggttaattggttagcgcagcctccatccgagttgcaagagaggaagacgcagtctcgccgaccctgttggcaaacagtaaaattgcaaaaaatagagtgttaggagtagttcccatacccttggggacaagaacctattatggagaaacgaactggcatcggaccctgggaaacaggcatctagcgtattaataggtcgctagaagacgggcaggcttcaccactaggtaacctgccgccgagagggaaaacctctgtgtggttgtgcgcacgtcgttgtcggaagaagttcaccatagtagttatttccttttcttttaggtagaagcacttgctccagagggcgcatacctctggttaaacaaaattgtcccctgagagtagaaaactcaggttaataaaacaggagaatatttac

SEQ ID NO: 25 - RNA, 44 nt, Red clover necrotic mosaic virus
  source 1..44: mol_type = genomic RNA
gcgtaacctccatccgagttgcaagagagggaaacgcagtctcg

SEQ ID NO: 26 - RNA, 120 nt, Red clover necrotic mosaic virus
  source 1..120: mol_type = genomic RNA
gaacaaacgttttaccggtttgtaaattggaattctcgaattcgccagtcgccttcttcgtatcaaggatctggaatttgaacggtatctttatcattttaatttctacttttcgtacca

SEQ ID NO: 27 - RNA, 473 nt, Red clover necrotic mosaic virus
  source 1..473: mol_type = genomic RNA
aatgccctagcgtaacctccatccgagttgcaagagagggaaacgcagtctcgttggttaattggttagcgcagcctccatccgagttgcaagagaggaagacgcagtctcgccgaccctgttggcaaacagtaaaattgcaaaaaatagagtgttaggagtagttcccatacccttggggacaagaacctattatggagaaacgaactggcatcggaccctgggaaacaggcatctagcgtattaataggtcgctagaagacgggcaggcttcaccactaggtaacctgccgccgagagggaaaacctctgtgtggttgtgcgcacgtcgttgtcggaagaagttcaccatagtagttatttccttttcttttaggtagaagcacttgctccagagggcgcatacctctggttaaacaaaattgtcccctgagagtagaaaactcaggttaataaaacaggagaatatttac

SEQ ID NO: 28 - RNA, 82 nt, Sweet clover necrotic mosaic virus
  source 1..82: mol_type = genomic RNA
ataaaaatctcgctcttcaaagactaattgaatccaagcaagcaaagactgtaagactacgatcaacatagagagatttgag

SEQ ID NO: 29 - RNA, 397 nt, Pestivirus C
  source 1..397: mol_type = genomic RNA
tatacgaggttagttcattctcgtatgcatgattggacaaattaaaatttcaatttggatcagggcctccctccagcgacggccgaactgggctagccatgcccacagtaggactagcaaacggagggactagccgtagtggcgagctccctgggtggtctaagtcctgagtacaggacagtcgtcagtagttcgacgtgagcagaagcccacctcgatatgctatgtggacgagggcatgcccaagacacaccttaaccctagcgggggtcgctagggtgaaatcacaccacgtgatgggagtacgacctgatagggtgctgcagaggcccactattaggctagtataaaaatctctgctgtacatggcactgagtggtaagctaacactccaatt

SEQ ID NO: 30 - RNA, 230 nt, Hepacivirus C
  source 1..230: mol_type = genomic RNA
ctgtttttttttttttttttttttttttttttttttttttttttttttttctctttcctaactttcctattatcttatatccttcttaaggtggctccatcttagccctagtcacggctagctgtgaaaggtccgtgagccgcatgactgcagagagtgctgatactggcctctctgcagatcatgtaaccccgcggggcctcttcgggggtctcgcggggttttttgct

SEQ ID NO: 31 - RNA, 43 nt, Potato leafroll virus
  source 1..43: mol_type = genomic RNA
tacactaatcccagagttgcaagcatgggaagttcaagcctcg

SEQ ID NO: 32 - RNA, 272 nt, Pestivirus A
  source 1..272: mol_type = genomic RNA
taatgaggggggtagcaacagtggtgagttcgttggatggcttaagccctgagtacagggtagtcgtcagtggttcgacgccttggttaaaaggtctcgaaatgccacgtggacgagggcacgcccaaagcacatcttaacctgagcgggggtcgctcagcgaaaacagtttaaccaactgctacgaatacagcctgatagggtgctgcagaggcccactgtattgctactaaaaatctctgctgtacatggcacgccgaactagctcagca

SEQ ID NO: 33 - RNA, 96 nt, Opium poppy mosaic virus
  source 1..96: mol_type = genomic RNA
aagcttagtcgcaggggttacacctccttgaggtattctggcaaatatcgggtattctggcaaatatcgccatgttataacatgggtgggtttaga

SEQ ID NO: 34 - RNA, 271 nt, Homo sapiens
  source 1..271: mol_type = genomic RNA
aggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcagcaccaaagcagccataaacaatatgtaaataaacagatgtggctgtattccagtacaactttacctacaaaaacaggcatcagaccagcttgccaacttgtggcatagactgtttgctactaag

SEQ ID NO: 35 - RNA, 433 nt, Zika virus
  source 1..433: mol_type = genomic RNA
caccagtcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtctcccggg

SEQ ID NO: 36 - RNA, 43 nt, Red clover necrotic mosaic virus
  source 1..43: mol_type = genomic RNA
gcgtaacctccatccgagttgcaagagagggaaacgcagtctc

SEQ ID NO: 37 - RNA, 129 nt, Red clover necrotic mosaic virus
  source 1..129: mol_type = genomic RNA
ataatggacaaacgttttaccggtttgtaaataggagcacacaagctcgctagtcgcccccttcgtatcggggatctagagattaaacggtatacctgtcattttaaatttctacttttcgtaccagtc

SEQ ID NO: 38 - RNA, 29 nt, Red clover necrotic mosaic virus
  source 1..29: mol_type = genomic RNA
tacctagtcgtataacggctaggtacccc